(19) United States
(12) Patent Application Publication
(10) Pub. No.: US 2007/0299109 A1
(43) Pub. Date: Dec. 27, 2007

(54) Dimeric Compounds Of Piperidine, Piperazine Or Morpholine Or Their 7-Membered Analogs Suitable For The Treatment Of Neurodegenerative Disorders

(76) Inventors: Miroslav Cik, Boechout (BE); Gaston Stanislas Marcella Diels, Ravels (BE); Guy Rosalia Eugeen Van Lommen, Berlaar (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/632,510

(22) PCT Filed: Jul. 13, 2005
(86) PCT No.: PCT/EP05/53345

§ 371 Date: Jan. 16, 2007

Related U.S. Application Data
(60) Provisional application No. 60/588,446, filed on Jul. 16, 2004.

(30) Foreign Application Priority Data
Jul. 16, 2004 (EP) 04103413.3

(51) Int. Cl. A61K 31/445; A61P 25/00; A61P 25/16; A61P 25/18; A61P 25/28; A61P 43/00; C07D 221/00

(52) U.S. Cl. 514/316; 546/186; 546/187

(57) ABSTRACT

Formula (I″), the N-oxide forms, the pharmaceutically acceptable addition salts and the stereochemically isomeric forms thereof.

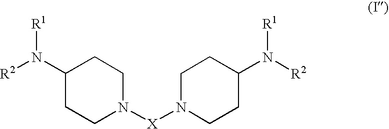

Neurotrophins, such as nerve growth factor (NGF), brain derived growth factor (BDNF), neurotrophic factor 3 (NT3) and neurotrophic factor 4 (NT4) mediate the survival, differentiation, growth and apoptosis of neurons. They bind to two structurally unrelated cell surface receptors, tropomyosin related kinase (Trk) receptors and p75 neurotrophin receptor (p75^{NTR}) (Kaplan D. R. and Miller F. D. (2000) Current Opinion in Neurobiology 10, 381-391). By activating those two type of receptors, neurotrophins mediate both, positive and negative survival signals. NGF binds with high affinity to TrkA, BDNF has high affinity for TrkB, NT-3 binds preferentially to TrkC. Binding of neurotrophins to Trk receptors is necessary for neurotrophic activity. P75^{NTR}, a member of TNF receptor superfamily was first neurotrophin receptor to be described. It binds all neurotrophins with similar affinity. P75^{NTR} was first described as a positive modulator of TrkA activity. Their co-expression lead to an increase of NGF affinity for TrkA receptors, NGF-mediated TrkA activation and ligand specificity. P75^{NTR} can also signal on it own and promote cell death in a variety of cell types. (Coulson E. J., Reid K., and Bartlett P. F. (1999) Molecular Neurobiology 20, 29-44).

Neurotrophins and Possible Therapeutical Relevance

Neurotrophins have a well established role in regulating the survival, differentiation and maintenance of functions of specific and sometimes overlapping neuronal populations. Besides these roles of neurotrophins during embryonic development and adulthood, there is increasing evidence that neurotrophins are involved in processes of neuronal plasticity. These studies suggest several potential therapeutic application. It has been shown that neurotrophins can protect and rescue certain neuronal populations in in vitro and in vivo models of various neurodegenerative diseases such as Alzheimer's disease, Parkinson's disease, Amyotrophic lateral sclerosis (ALS), stroke and peripheral neuropathies (Chao M. V. (2003) Nature Reviews Neuroscience 4, 299-309; Dawbarn D. and Allen S. J. (2003) Neuropathology & Applied Neurobiology 29, 211-230).

In addition, accumulating evidence in last few years shows that p75^{NTR} plays a key role in neuronal death that occurs in some of the major disorders of the CNS such as stroke, Alzheimer's, ALS, epilepsy, Spinal Cord Injury (SCI), Multiple Sclerosis (MS), Motor Neuron Disease (MND) and other neurodegenerative diseases (Park et al. (2000) Journal of Neuroscience 20, 9096-9103; Oh et al. (2000) Brain Research 853, 174-185; Lowry et al. (2001) Journal of Neuroscience Research 64, 11-17; Sedel et al. (1999) European Journal of Neuroscience 11, 3904-3912; Dowling et al. (1999) Neurology 53, 1676-1682) and only recently, NGF was found to play an important role in pain, in particular in post-operative pain after surgery (Zahn et al. 2004, The Journal of Pain 5(3); 157-163). For these reasons small molecules that enhance the activity of neurotrophins, or that have similar effects as neurotrophins, are of great interest (Massa et al. (2002) Journal of Molecular Neuroscience 19, 107-111; Saragovi and Burgess (1999) Expert Opinion on Therapeutic Patents 9, 737-751).

Experimental Evidence

Peripheral neurons derived from chick embryo dorsal root ganglia (DRG) are extensively used for in vitro characterizations of neurotrophic factors and other molecules with neurotrophic activities. The survival of chick DRG neurons can be supported by different neurotrophic factors, such as nerve growth factor (NGF) (Levi-Montalcini R. and Angeletti P. U. (1968) Physiological Reviews 48, 534-569) brain derived neurotrophic factor (Barde Y. A. et al. (1982) EMBO Journal 1, 549-553) and ciliary neurotrophic factor (CNTF) (Barbin G. et al. (1984) Journal of Neurochemistry 43, 1468-1478). Small molecules with the neurotrophic activity, such as K-252a and CEP-1347 also support the survival of DRG neurons (Borasio G. D. (1990) Neuroscience Letters 108, 207-212; Borasio G. D. et al. (1998) Neuroreport 9, 1435-1439). The primary culture of dissociated DRG neurons from chicken embryo at embryonic day 8-10 has been used successfully in a number of laboratories as a bioassay for neurotrophins. The assay determines the survival effect of compounds on DRG neurons and is based on a fluorimetric Calcein-AM measurement (He W. et al. (2002) Bioorganic & Medicinal Chemistry 10, 3245-3255). This assay, which addresses the functional response of neurons as a quantitative measure of survival, may have the advantage of few false positive.

HTS campaign using a primary culture of chicken DRG neurons, resulted in the identification of compounds with neurotrophic activity (neuronal survival). The most potent compounds identified belong to a series of “symmetrical compounds”.

This invention concerns compounds of formula (I)

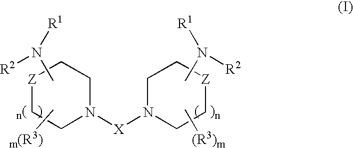

the N-oxide forms, the pharmaceutically acceptable addition salts and the stereochemically isomeric forms thereof, wherein
n represents 0, 1 or 2;
m represents 0, 1, 2 or 3;
Z represent C, N or O, in particular Z represents CH₂;
—X— represents C₂₋₄alkynyl, C₁₋₁₂alkyl optionally substituted with hydroxy or X represents a divalent radical of the formula 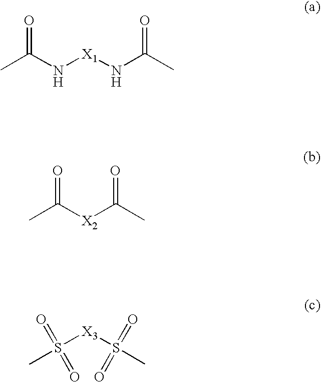
wherein; —X₁— represents C₁₋₁₂alkyl, phenyl or a divalent radical selected from the group consisting of 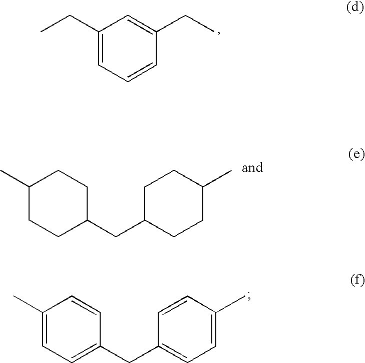
   —X₂— represents C₁₋₁₂alkyl, C₁₋₄alkyloxyC₁₋₄alkyl, phenyl or a divalent radical of formula 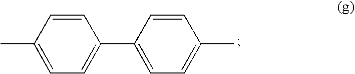
   —X₃— represents phenyl or a divalent radical selected from the group consisting of 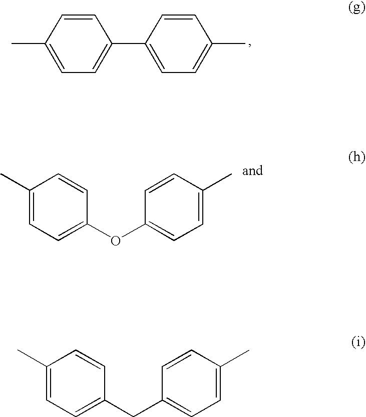
R¹ and R² each independently represents hydrogen, C₁₋₄alkyl, C₁₋₄alkyl-carbonyl-, Ar¹-carbonyl-, Het¹, Ar² or C₁₋₄alkyl-carbonyl- substituted with Het² or Ar³; or
R¹ and R² taken together with the nitrogen atom with which they are attached form a heterocycle selected from pyrimidinyl, indolyl, indolinyl, indazolyl, imidazolinyl, imidazolidinyl, benzoxazolyl, benzimidazolyl, quinazolinyl, quinolinyl or benzthiazolyl wherein said heterocycle is optionally substituted with one or where possible two or more substituents selected from the group consisting of carbonyl, Ar⁵, amino, mono- or di-substituted (C₁₋₄alkyl)-amino-, hydroxy, halo, polyhaloC₁₋₄alkyloxy-, C₁₋₄alkyl, C₁₋₄alkyloxycarbonyl- and phenyl;
R³ independently represents hydroxy or C₁₋₄alkyloxy-;
Het¹ represents a heterocycle selected from pyridinyl, indolinyl, benzimidazolyl, benzthiazolyl, thiazolyl, pyridinyl, benzisoxazolyl, benzoxazolyl, oxadiazolyl or thiadiazolyl wherein said Het¹ is optionally substituted with one or where possible two or more substituents selected from the group consisting of hydroxy, halo, Ar⁴, C₁₋₄alkyloxycarbonyl-, C₁₋₄alkyl-, C₁₋₄alkyloxy- and C₁₋₄alkyloxy- substituted with halo;
Het² represents a heterocycle selected from thiophenyl, furanyl, pyrrolyl, pyridinyl, thiazolyl, oxazolyl, pyridinyl, benzisoxazolyl, benzoxazolyl or thiadiazolyl wherein said Het² is optionally substituted with one or where possible two or more substituents selected from the group consisting of hydroxy, halo, Het⁴, C₁₋₄alkyloxycarbonyl-, C₁₋₄alkyl-, C₁₋₄alkyloxy- and C₁₋₄alkyloxy- substituted with halo;
Het³ represents a heterocycle selected from thiophenyl, furanyl, pyrrolyl, pyridinyl, thiazolyl, oxazolyl, pyridinyl, or thiadiazolyl;
Het⁴ represents a heterocycle selected from thiophenyl, furanyl, pyrrolyl, pyridinyl, thiazolyl, oxazolyl, pyridinyl, or thiadiazolyl wherein said Het⁴ is optionally substituted with one or where possible two or more substituents selected from the group consisting of hydroxy, halo, C₁₋₄alkyl- and C₁₋₄alkyloxy-;
Ar¹, Ar² and Ar³ each independently represent phenyl optionally substituted with halo, amino, Het³, C₁₋₄alkylcarbonyl-, C₁₋₄alkyl, C₁₋₄alkyloxy- or C₁₋₄alkyl substituted with one, two or three halo substituents; in particular Ar¹, Ar² and Ar³ each independently represent phenyl optionally substituted with halo, C₁₋₄alkyl or C₁₋₄alkyloxy-;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

As used herein before, the terms;
oxo or carbonyl refers to (═O) that forms a carbonyl moiety with the carbon atom to which it is attached;
halo is generic to fluoro, chloro, bromo and iodo;
C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like;
C₁₋₆alkyl is meant to include C₁₋₄-alkyl and the higher homologues thereof having 6 carbon atoms such as, for example hexyl, 1,2-dimethylbutyl, 2-methylpentyl and the like;
C₁₋₄alkyloxy defines straight or branched saturated hydrocarbon radicals having from 1 to 4 carbon atoms and 1 oxygen atom such as methoxy, ethoxy, propyloxy, butyloxy, 1-methylethyloxy, 2-methylpropyloxy and the like.

The heterocycles as mentioned in the above definitions and hereinafter, are meant to include all possible isomeric forms thereof, for instance triazolyl also includes 1,2,4-triazolyl and 1,3,4-triazolyl; oxadiazolyl includes 1,2,3-oxadiazolyl, 1,2,4-oxadiazolyl, 1,2,5-oxadiazolyl and 1,3,4-oxadiazolyl; thiadiazolyl includes 1,2,3-thiadiazolyl, 1,2,4-thiadiazolyl, 1,2,5-thiadiazolyl and 1,3,4-thiadiazolyl.

Further, the heterocycles as mentioned in the above definitions and hereinafter may be attached to the remainder of the molecule of formula (I) through any ring carbon or heteroatom as appropriate. Thus, for example, when the heterocycle is imidazolyl, it may be a 1-imidazolyl, 2-imidazolyl, 4-imidazolyl and 5-imidazolyl; when it is thiazolyl, it may be 2-thiazolyl, 4-thiazolyl and 5-thiazolyl; when it is benzothiazolyl, it may be 2-benzothiazolyl, 4-benzothiazolyl, 5-benzothiazolyl, 6-benzothiazolyl and 7-benzothiazolyl.

The pharmaceutically acceptable addition salts as mentioned hereinabove are meant to comprise the therapeutically active non-toxic acid addition salt forms, which the compounds of formula (I), are able to form. The latter can conveniently be obtained by treating the base form with such appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-amninosalicylic, pamoic and the like acids.

The pharmaceutically acceptable addition salts as mentioned hereinabove are meant to comprise the therapeutically active non-toxic base addition salt forms which the compounds of formula (I), are able to form. Examples of such base addition salt forms are, for example, the sodium, potassium, calcium salts, and also the salts with pharmaceutically acceptable amines such as, for example, ammonia, alkylamines, benzathine, N-methyl-D-glucamine, hydrabamine, amino acids, e.g. arginine, lysine.

Conversely said salt forms can be converted by treatment with an appropriate base or acid into the free acid or base form.

The term addition salt as used hereinabove also comprises the solvates which the compounds of formula (I), as well as the salts thereof, are able to form. Such solvates are for example hydrates, alcoholates and the like.

The term stereochemically isomeric forms as used hereinbefore defines the possible different isomeric as well as conformational forms which the compounds of formula (I), may possess. Unless otherwise mentioned or indicated, the chemical designation of compounds denotes the mixture of all possible stereochemically and conformationally isomeric forms, said mixtures containing all diastereomers, enantiomers and/or conformers of the basic molecular structure. All stereochemically isomeric forms of the compounds of formula (I), both in pure form or in admixture with each other are intended to be embraced within the scope of the present invention.

The N-oxide forms of the compounds of formula (I), are meant to comprise those compounds of formula (I) wherein one or several nitrogen atoms are oxidized to the so-called N-oxide.

A particular group of the compounds of the present invention consist of those compounds of formula (I) wherein one or more of the following restrictions apply;
—X— represents C₂₋₄alkynyl, C₁₋₁₂alkyl optionally substituted with hydroxy or X represents a divalent radical of the formula 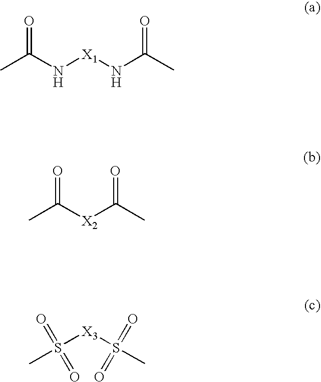
wherein; —X₁— represents C₁₋₁₂alkyl, phenyl or a divalent radical selected from the group consisting of 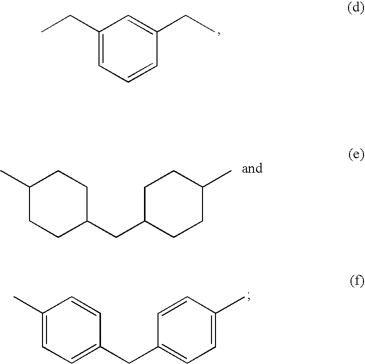
   —X₂— represents C₁₋₁₂alkyl, C₁₋₄alkyloxyC₁₋₄alkyl, phenyl or a divalent radical of formula 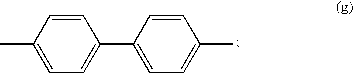
   —X₃— represents phenyl or a divalent radical selected from the group consisting of 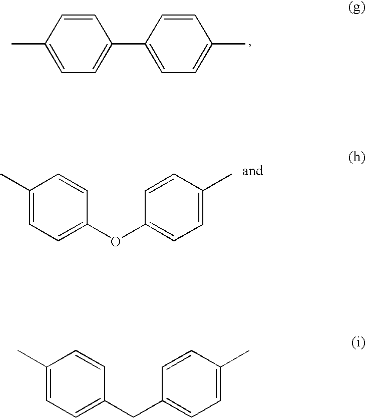
n represents 1;
m represents 0, 1 or 2; in particular m represents 0;
R¹ and R² each independently represent hydrogen, C₁₋₄alkyl, Ar¹-carbonyl, Het¹, Ar² or C₁₋₄alkylcarbonyl optionally substituted with Het² or Ax³; or
R¹ and R² taken together with the nitrogen atom to which they are attached form a heterocycle selected from indolyl, indolinyl, benzimidazolyl, benzthiazolyl, benzisoxazolyl or oxodiazolyl wherein said heterocycle is optionally substituted with one or where possible two or more substituents selected from the group consisting of hydroxy, C₁₋₄alkyl, C₁₋₄alkyloxycarbonyl, carbonyl, Ar⁵ and halo; in particular R¹ and R² taken together with the nitrogen atom with which they are attached form a heterocycle selected from indolinyl, benzimidazolyl, or benzthiazolyl wherein said heterocycle is optionally substituted with one or where possible two or more substituents selected from the group consisting of hydroxy, halo, C₁₋₄alkyl, C₁₋₄alkyloxycarbonyl- and phenyl;
Het¹ represents a heterocycle selected from pyridinyl, indolinyl, indolyl, benzthiazolyl, benzimidazolyl, thiazolyl, thiadiazolyl or benzisoxazolyl wherein said Het¹ is optionally substituted with one or where possible two or more substituents selected from the group consisting of hydroxy, halo, Ar⁴, C₁₋₄alkyloxycarbonyl-, C₁₋₄alkyl and C₁₋₄alkyloxy-, said C₁₋₄alkyloxy- being optionally substituted with halo; in particular Het¹ represents a heterocycle selected from pyridinyl, indolinyl, benzimidazolyl, benzthiazolyl, thiazolyl, or thiadiazolyl wherein said Het¹ is optionally substituted with one or where possible two or more substituents selected from the group consisting of hydroxy, halo, Ar⁴, C₁₋₄alkyloxycarbonyl-, C₁₋₄alkyl-, C₁₋₄alkyloxy- and C₁₋₄alkyloxy- substituted with halo;
Het² represents a heterocycle selected from thiophenyl, furanyl, pyrrolyl, pyridinyl, thiazolyl, oxazolyl, pyridinyl, or thiadiazolyl;
Ar¹, Ar² and Ar³ each independently represent phenyl optionally substituted with halo, C₁₋₄alkyl, C₁₋₄alkyloxy- or C₁₋₄alkyl substituted with one, two or three halo substituents; in particular Ar¹, Ar² and Ar³ each independently represent phenyl optionally substituted with halo, C₁₋₄alkyl or C₁₋₄alkyloxy-; in particular Ar¹ represents phenyl optionally substituted with halo, amino, C₁₋₄alkyl or C₁₋₄alkyloxy-; Ar² represents phenyl optionally substituted with halo, C₁₋₄alkyl, C₁₋₄alkyloxy- or Het³-C₁₋₄alkyl-carbonyl-; in particular Ar² represents phenyl substituted with halo; and Ar³ represents phenyl optionally substituted with halo, C₁₋₄alkyl or C₁₋₄alkyloxy-;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

An interesting group of compounds are those compounds of formula (I′)

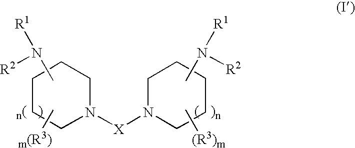

the N-oxide forms, the pharmaceutically acceptable addition salts and the stereochemically isomeric forms thereof, wherein
n represents 1 or 2;
m represents 0, 1, 2 or 3;
—X— represents C₂₋₄alkynyl, C₁₋₁₂alkyl optionally substituted with hydroxy or X represents a divalent radical of the formula 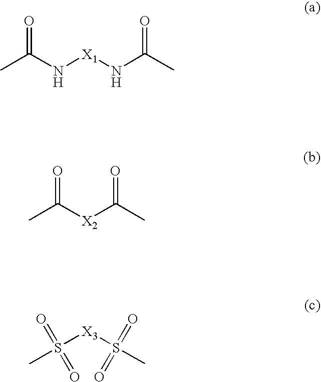
wherein; —X₁— represents C₁₋₁₂alkyl, phenyl or a divalent radical selected from the group consisting of 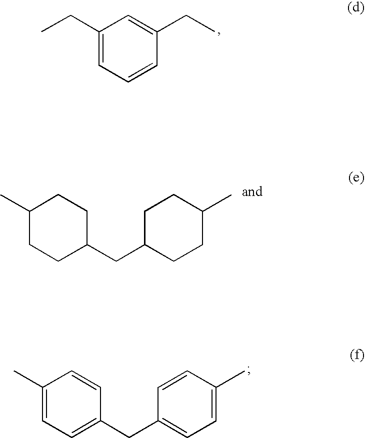
   —X₂— represents C₁₋₁₂alkyl, C₁₋₄alkyloxyC₁₋₄alkyl, phenyl or a divalent radical of formula 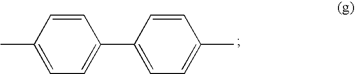
   —X₃— represents phenyl or a divalent radical selected from the group consisting of 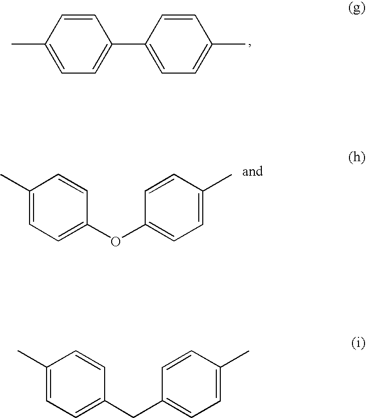
R¹ and R² each independently represents hydrogen, C₁₋₄alkyl, C₁₋₄alkyl-carbonyl-, Ar¹-carbonyl-, Het¹, Ar² or C₁₋₄alkyl-carbonyl- substituted with Het² or Ar³; or
R¹ and R² taken together with the nitrogen atom with which they are attached form a heterocycle selected from pyrimidinyl, indolyl, indolinyl, indazolyl, imidazolinyl, imidazolidinyl, benzoxazolyl, benzimidazolyl, quinazolinyl, quinolinyl or benzthiazolyl wherein said heterocycle is optionally substituted with one or where possible two or more substituents selected from the group consisting of carbonyl, Ar⁵, amino, mono- or di-substituted (C₁₋₄alkyl)-amino-, hydroxy, halo, polyhaloC₁₋₄alkyloxy-, C₁₋₄alkyl, C₁₋₄alkyloxycarbonyl- and phenyl;
R³ independently represents hydroxy or C₁₋₄alkyloxy-;
Het¹ represents a heterocycle selected from pyridinyl, indolinyl, benzimidazolyl, benzthiazolyl, thiazolyl, pyridinyl, benzisoxazolyl, benzoxazolyl, oxadiazolyl or thiadiazolyl wherein said Het¹ is optionally substituted with one or where possible two or more substituents selected from the group consisting of hydroxy, halo, Ar⁴, C₁₋₄alkyloxycarbonyl-, C₁₋₄alkyl-, C₁₋₄alkyloxy- and C₁₋₄alkyloxy- substituted with halo;
Het² represents a heterocycle selected from thiophenyl, furanyl, pyrrolyl, pyridinyl, thiazolyl, oxazolyl, pyridinyl, benzisoxazolyl, benzoxazolyl or thiadiazolyl wherein said Het² is optionally substituted with one or where possible two or more substituents selected from the group consisting of hydroxy, halo, Het⁴, C₁₋₄alkyloxycarbonyl-, C₁₋₄alkyl-, C₁₋₄alkyloxy- and C₁₋₄alkyloxy- substituted with halo;
Het³ represents a heterocycle selected from thiophenyl, furanyl, pyrrolyl, pyridinyl, thiazolyl, oxazolyl, pyridinyl, or thiadiazolyl;
Het⁴ represents a heterocycle selected from thiophenyl, furanyl, pyrrolyl, pyridinyl, thiazolyl, oxazolyl, pyridinyl, or thiadiazolyl wherein said Het⁴ is optionally substituted with one or where possible two or more substituents selected from the group consisting of hydroxy, halo, C₁₋₄alkyl- and C₁₋₄alkyloxy-;
Ar¹, Ar² and Ar³ each independently represent phenyl optionally substituted with halo, amino, Het³, C₁₋₄-alkylcarbonyl-, C₁₋₄alkyl, C₁₋₄alkyloxy- or C₁₋₄-alkyl substituted with one, two or three halo substituents; in particular Ar¹, Ar² and Ar³ each independently represent phenyl optionally substituted with halo, C₁₋₄alkyl or C₁₋₄alkyloxy-;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

Also of interest are those compounds of formula (I) wherein one or more of the following restrictions apply;
n represents 1;
m represents 0;
R¹ and R² each independently represent hydrogen, C₁₋₄alkyl, Ar¹-carbonyl, Het¹, Ar² or C₁₋₄alkylcarbonyl optionally substituted with Het² or Ar³; or
R¹ and R² taken together with the nitrogen atom to which they are attached form a heterocycle selected from indolyl, indolinyl, benzimidazolyl, benzthiazolyl, benzisoxazolyl or oxodiazolyl wherein said heterocycle is optionally substituted with one or where possible two or more substituents selected from the group consisting of hydroxy, C₁₋₄alkyl, carbonyl, C₁₋₄alkyloxycarbonyl-, Ar⁵and halo;
Het¹ represents a heterocycle selected from pyridinyl, indolinyl, indolyl, benzthiazolyl, benzimidazolyl, thiazolyl, thiadiazolyl or benzisoxazolyl wherein said Het¹ is optionally substituted with one or where possible two or more substituents selected from the group consisting of halo, Ar⁴, C₁₋₄alkyloxycarbonyl-, C₁₋₄alkyl and C₁₋₄alkyloxy-, said C₁₋₄alkyloxy- being optionally substituted with halo;
Het² represents a heterocycle selected from thiophenyl, furanyl, pyrrolyl, pyridinyl, thiazolyl, oxazolyl, pyridinyl, or thiadiazolyl;
Ar¹, Ar² and Ar³ each independently represent phenyl optionally substituted with halo, C₁₋₄alkyl, C₁₋₄alkyloxy- or C₁₋₄alkyl substituted with one, two or three halo substituents;
Ar⁴ represents phenyl optionally substituted with halo, C₁₋₄alkyl, C₁₋₄alkyloxy- or C₁₋₄-alkyl substituted with one, two or three halo substituents;
Ar⁵ represents phenyl optionally substituted with C₁₋₄alkyloxy- or C₃₋₆cycloalkyloxy-.

A further group of compounds of formula (I) consist of those compounds of formula (I) wherein one or more of the following restrictions apply;
n represents 1;
m represents 0;
Z represents CH₂;
R¹ and R² each independently represent hydrogen, C₁₋₄alkyl, Ar¹-carbonyl, Het¹, Ar² or C₁₋₄alkylcarbonyl optionally substituted with Het² or Ar³; or
R¹ and R² taken together with the nitrogen atom to which they are attached form a heterocycle selected from indolyl, indolinyl, benzimidazolyl, benzthiazolyl, benzisoxazolyl or oxodiazolyl wherein said heterocycle is optionally substituted with one or where possible two or more substituents selected from the group consisting of hydroxy, C₁₋₄alkyl, carbonyl, C₁₋₄alkyloxycarbonyl-, Ar⁵ and halo;
Het¹ represents a heterocycle selected from pyridinyl, indolinyl, indolyl, benzthiazolyl, benzimidazolyl, thiazolyl, thiadiazolyl or benzisoxazolyl wherein said Het¹ is optionally substituted with one or where possible two or more substituents selected from the group consisting of halo, Ar⁴, C₁₋₄alkyloxycarbonyl-, C₁₋₄alkyl and C₁₋₄alkyloxy-, said C₁₋₄alkyloxy- being optionally substituted with halo;
Het² represents a heterocycle selected from thiophenyl, furanyl, pyrrolyl, pyridinyl, thiazolyl, oxazolyl, pyridinyl, or thiadiazolyl;
Ar¹, Ar² and Ar³ each independently represent phenyl optionally substituted with halo, C₁₋₄alkyl, C₁₋₄alkyloxy- or C₁₋₄alkyl substituted with one, two or three halo substituents;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

Another interesting group of compounds according to the invention are those compounds of formula (I) or formula (I′) wherein one or more of the following restrictions apply;
n represents 1;
m represents 0;
Z represents C, in particular CH₂ for those compounds of formula (I);
R¹ and R² each independently represents hydrogen, C₁₋₄alkyl, Ar¹-carbonyl-, Het¹, Ar² or C₁₋₄alkylcarbonyl- substituted with Het² or Ar³; or
R¹ and R² taken together with the nitrogen atom to which they are attached form a heterocycle selected from indolyl, indolinyl, or benzimidazolyl wherein said heterocycle is optionally substituted with one or where possible two or more substituents selected from the group consisting of carbonyl, hydroxy or halo;
Het¹ represents a heterocycle selected from pyridinyl, indolinyl, benzthiazolyl, thiazolyl, or thiadiazolyl, wherein said Het¹ is optionally substituted with one or where possible two or more substituents selected from the group consisting of halo, Ar⁴, C₁₋₄alkyloxycarbonyl- and C₁₋₄alkyloxy- substituted with halo;
Het² represents thiophenyl;
Ar¹ represents phenyl optionally substituted with halo or C₁₋₄alkyloxy-;
Ar² represents phenyl optionally substituted with halo or C₁₋₄-alkyloxy;
Ar³ represents phenyl optionally substituted with halo or C₁₋₄alkyl; or
Ar⁴ represents phenyl optionally substituted with C₁₋₄alkyl-.

Also of interest are those compounds of formula (I) or (I′) wherein;
m represents 0;
Z represents C or N, in particular C, more in particular CH₂ for those compounds of formula (I);
n represents 1;
—X— represents C₂₋₄alkynyl, C₁₋₁₂alkyl optionally substituted with hydroxy or —X— represents a divalent radical of the formula (a), (b) or (c) as defined hereinbefore
wherein; —X₁— represents C₁₋₁₂alkyl or a divalent radical selected from (d) or (e) as defined for the compounds of formula (I) hereinbefore;
   —X₂— represents C₁₋₁₂alkyl, C₁₋₄alkyloxyC₁₋₄alkyl, phenyl or a divalent radical of formula (g) as defined for the compounds of formula (I) hereinbefore;
   —X₃— represents phenyl or a divalent radical selected from the (g), (h) and (i) as defined for the compounds of formula (I) hereinbefore;
R¹ and R² each independently represent hydrogen, C₁₋₄alkyl or R¹ and R² taken together with the nitrogen atom to which they are attached form a heterocycle selected from indolyl, indolinyl or benzimidazolyl wherein said heterocycle is optionally substituted with one or where possible two or more substituents selected from the group consisting of carbonyl, hydroxy or halo;
Het¹ represents a heterocycle selected from pyridinyl, indolinyl or benzthiazolyl wherein said Het¹ is optionally substituted with halo, Ar⁴ or polyhaloC₁₋₄alkyloxy-; Het² represents thiophenyl;
Ar¹ represents phenyl optionally substituted with halo or C₁₋₄alkyloxy-;
Ar² represents phenyl optionally substituted with halo or C₁₋₄alkyloxy;
Ar³ represents phenyl optionally substituted with halo or C₁₋₄alkyl; or
Ar⁴ represents phenyl optionally substituted with C₁₋₄alkyl-.

It is accordingly an object of the present invention to provide the compounds of formula (I″)

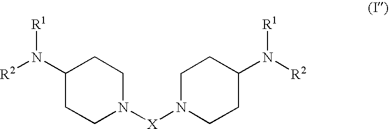

the N-oxide forms, the pharmaceutically acceptable addition salts and the stereochemically isomeric forms thereof, wherein
—X— represents C₂₋₄alkynyl, C₁₋₁₂alkyl optionally substituted with hydroxy or X represents a divalent radical of the formula 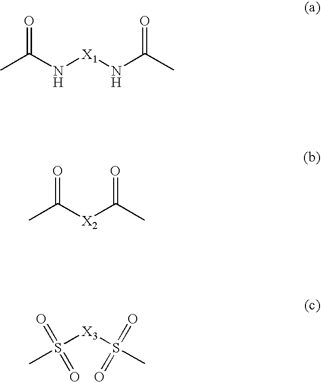
wherein; —X₁— represents C₁₋₁₂alkyl, phenyl or a divalent radical selected from the group consisting of 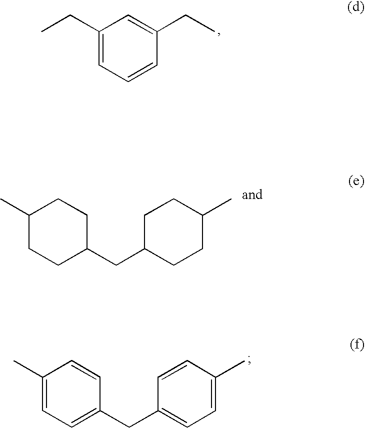
   —X₂— represents C₁₋₁₂alkyl, C₁₋₄alkyloxyC₁₋₄alkyl, phenyl or a divalent radical of formula 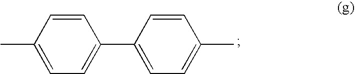
   —X₃— represents phenyl or a divalent radical selected from the group consisting of 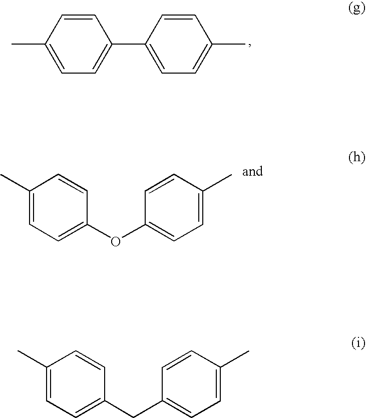
R¹ and R² each independently represents hydrogen, C₁₋₄alkyl, C₁₋₄alkyl-carbonyl-, Ar¹-carbonyl-, Het¹, Ar² or C₁₋₄alkyl-carbonyl- substituted with Het² or Ar³; or
R¹ and R² taken together with the nitrogen atom with which they are attached form a heterocycle selected from indolinyl, benzimidazolyl, or benzthiazolyl wherein said heterocycle is optionally substituted with one or where possible two or more substituents selected from the group consisting of hydroxy, halo, C₁₋₄alkyl, C₁₋₄alkyloxycarbonyl- and phenyl;
Het¹ represents a heterocycle selected from pyridinyl, indolinyl, benzimidazolyl, benzthiazolyl, thiazolyl, pyridinyl, or thiadiazolyl wherein said Het¹ is optionally substituted with one or where possible two or more substituents selected from the group consisting of hydroxy, halo, Ar⁴, C₁₋₄alkyloxycarbonyl-, C₁₋₄alkyl-, C₁₋₄alkyloxy- and C₁₋₄alkyloxy- substituted with halo;
Het² represents a heterocycle selected from thiophenyl, furanyl, pyrrolyl, pyridinyl, thiazolyl, oxazolyl, pyridinyl, or thiadiazolyl;
Ar¹ represents phenyl optionally substituted with halo, C₁₋₄alkyl or C₁₋₄alkyloxy-;
Ar² represents phenyl optionally substituted with halo, C₁₋₄alkyl or C₁₋₄alkyloxy-; in particular Ar² represents phenyl substituted with halo;
Ar³ represents phenyl optionally substituted with halo, C₁₋₄alkyl or C₁₋₄alkyloxy-;
Ar⁴ represents phenyl optionally substituted with halo, C₁₋₄alkyl or C₁₋₄alkyloxy-.

In a further embodiment the compounds of the present invention consist of those compounds of formula (I) wherein n represents 1, m represents 0, Z represents C, in particular CH₂ and the NR¹R² substituent is in the para position vis-á-vis the N-atom of the piperidine ring. Said NR¹R² substituent preferably consists of benzthiazolyl optionally substituted with halo or phenyl or R¹ and R² each independently represent hydrogen, Het¹, Ar²,

C₁₋₄alkyl or Ar¹-carbonyl-, in particular either R¹ or R² represents hydrogen, C₁₋₄alkyl or methylphenylcarbonyl and R² or R¹ respectively, represents pyridinyl or benzthiazolyl.

In an even further embodiment the compounds of the present invention are selected from the compounds according to formulae (A)-(O) below:

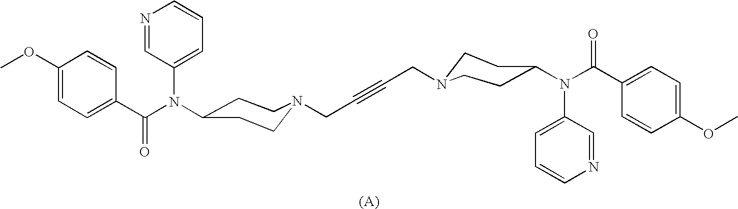

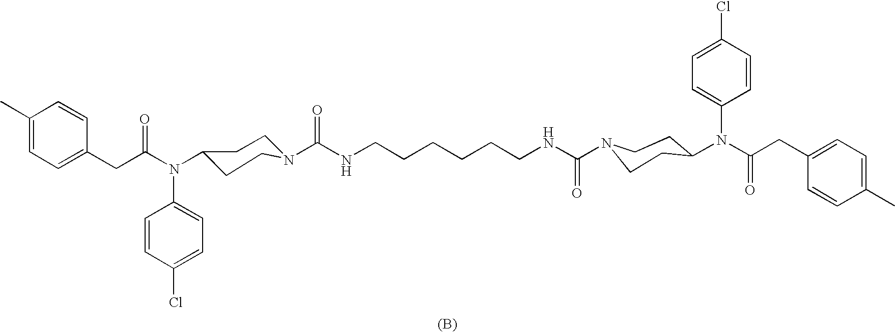

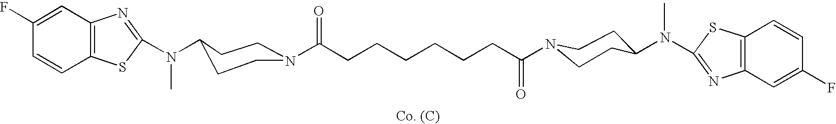

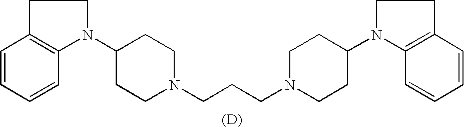

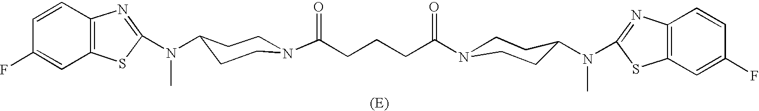

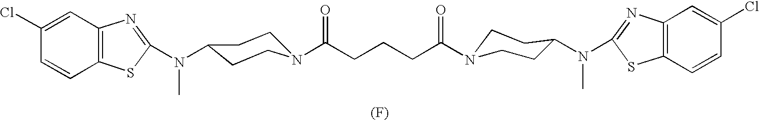

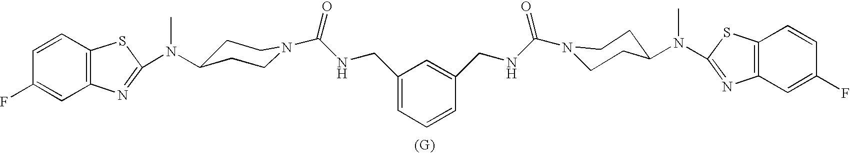

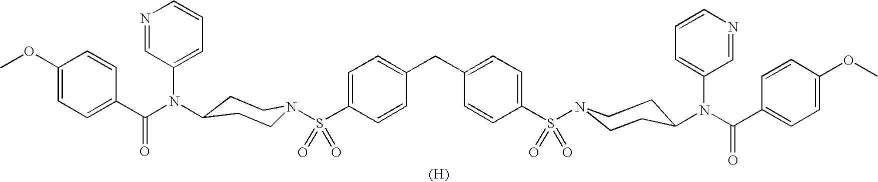

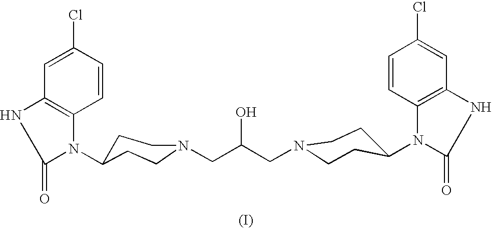

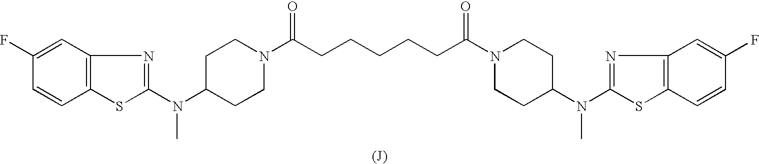

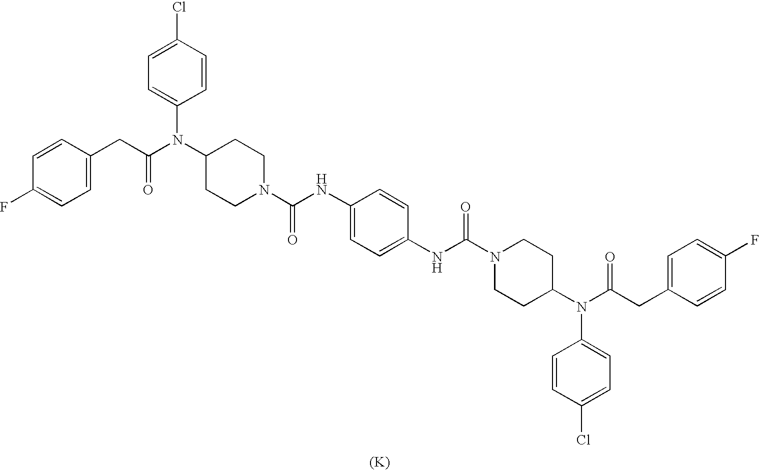

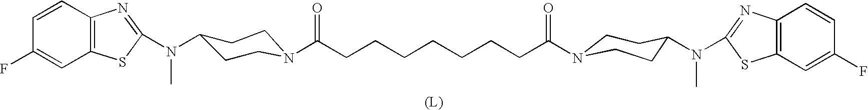

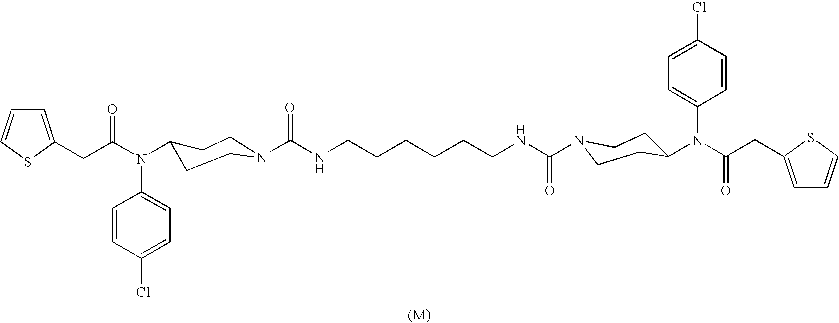

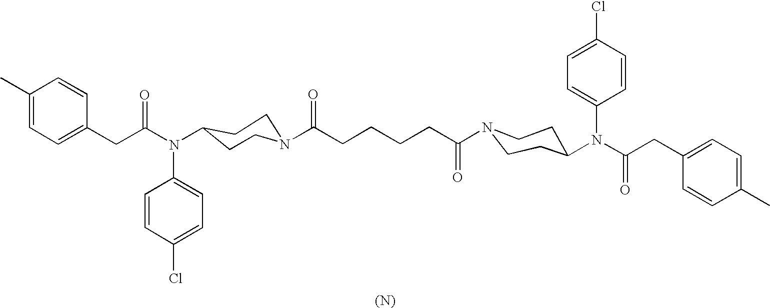

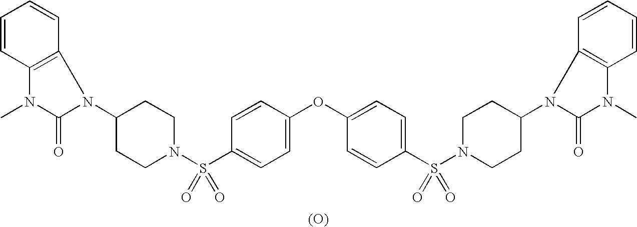

The dimeric compounds of this invention can be prepared by any of several standard synthetic processes commonly used by those skilled in the art of organic chemistry and described for instance in; “Introduction to organic chemistry” Streitweiser and Heathcock—Macmillan Publishing Co., Inc.—second edition—New York.

In general, for those compounds where X represents a C₂₋₄alkynyl or an optionally substituted C₁₋₁₂alkyl, the dimeric compounds are obtained by a nucleofilic substitution reaction between the appropriate secondary amine (i) with an alkylhalide (scheme 1) under basic reaction conditions, such as for example described in “Introduction to organic chemistry” Streitweiser and Heathcock—Macmillan Publishing Co., Inc.—second edition—New York, page 742—section 24.6.

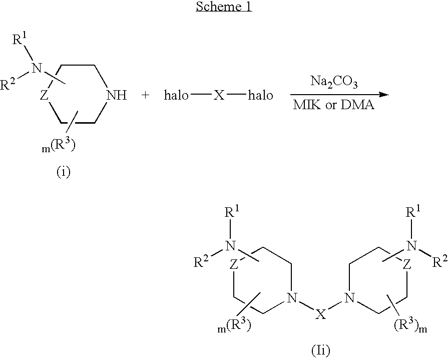

Wherein m, Z, X, R¹, R² and R³ are defined as for the compounds of formula (I)

For those compounds where X represents a divalent radical of formula (a) the urea derivatives of formula (Iii) are prepared by reacting the appropriate secondary amine with an isocyanate of general formula (ii) under art known conditions such as for example described in “Advanced Organic Chemistry” Jerry March—John Wiley & Sons, Inc.—third edition—New York, page 802—section 6-17. 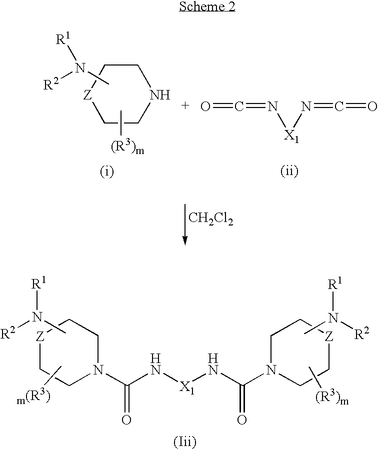
Wherein m, Z, X₁, R¹,R²and R³ are defined as for the compounds of formula (I)

Those compounds where X represents a divalent radical of formula (b), the amide derivatives of formula (Iiii) are prepared by reacting the appropriate secondary amine with an acylhalide of general formula (iii) under art known conditions such as for example described in “Advanced Organic Chemistry” Jerry March—John Wiley & Sons, Inc.—third edition—New York, page 370—section 0-54. Alternatively the amide derivatives of formula (Iiii) are obtained by acylation of the appropriate secondary amine with an bisanhydride of general formula (iv) under art known conditions such as for example described in “Advanced Organic Chemistry” Jerry March—John Wiley & Sons, Inc.—third edition—New York, page 371—section 0-55, or by acylation of the appropriate secondary amine with an ester of general formula (v) under art known conditions such as for example described in “Advanced Organic Chemistry” Jerry March—John Wiley & Sons, Inc.—third edition—New York, page 375—section 0-57. 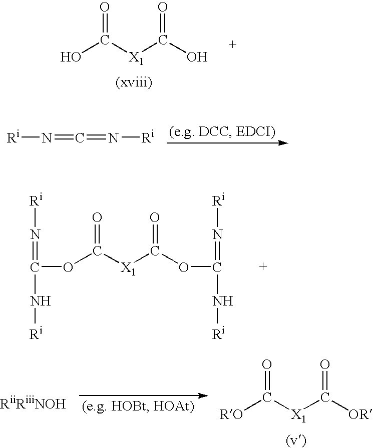
Wherein X₁ is defined as for the compounds of formula (I) and R¹ represents RⁱⁱRⁱⁱⁱN—

In a further alternative the active ester intermediates of formula (v′) (see scheme 3) are obtained by reaction of the appropriate secondary amine with a carboxylic acid (xviii) in the presence of reagantia, i.e. coupling reagents such as for example N,N′-Dicyclohexylcarbodiimide (DCC), N-(3-Dimethylaminopropyl)-N′-ethylcarbodiimide hydrochloride (EDCI), (Benzotriazol-1-yloxy)tripyrrolidinophosphonium hexafluorophosphate (PyBOP) or O-(Benzotriazol-1-yl)-N,N,N′,N′-tetramethyluronium hexafluorophosphate (HBTU), which in a first step convert the carboxylic acid in an activated form. This reaction is preferably performed in the presence of a further hydroxylamine additive, such as 1-hydroxybenzotriazole (HOBt) or 7-aza-1-hydroxybenzotriazole (HOAt), to prevent dehydration of the carboxamide residues thus obtained. 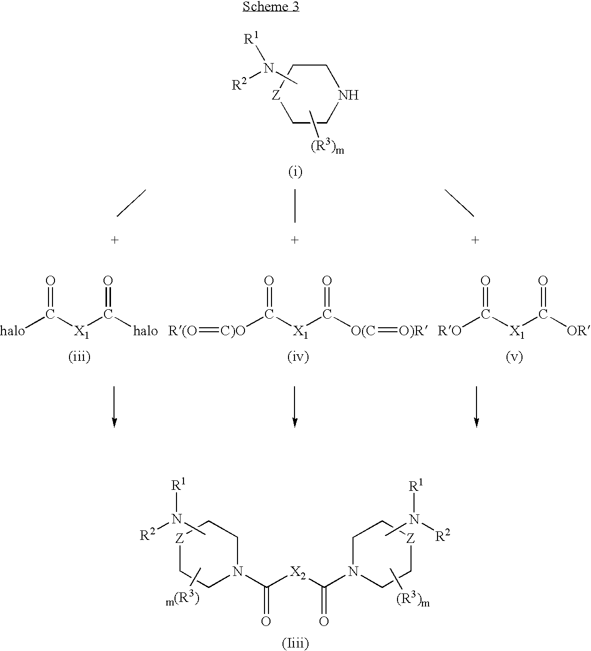
Wherein m, Z, X₂, R¹, R² and R³ are defined as for the compounds of formula (I), R′ represents a C₁₋₄alkyl, preferably ethyl and wherein halo represents a halogen such as for example Cl, Br and I

Finally, the sulfonamide derivative of formula (Iiv) where X represents a divalent radical of formula (c) are generally prepared by a nucleophilic substitution reaction between the appropriate secondary amine and a sulfonylhalide, preferably a sulfonylchloride of general formula (vi) under art known conditions such as for example described in “Advanced Organic Chemistry” Jerry March—John Wiley & Sons, Inc.—third edition—New York, page 445—section 0-119. 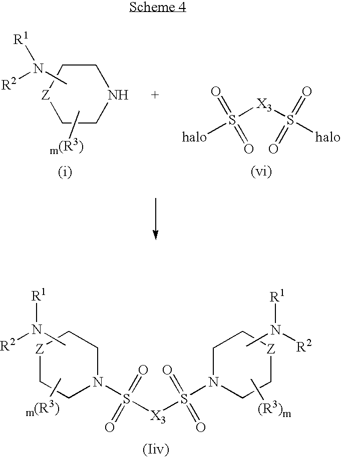
Wherein m, Z, X₃, R¹, R²and R³ are defined as for the compounds of formula (I) and wherein halo represents a halogen such as for example Cl, Br and I, preferably Cl

The appropriate secondary amines as used hereinbefore are either commercially available or in a particular embodiment, prepared departing from 4-piperidone or 4-amino-piperidine wherein the N-atom of the piperidine ring is shielded by means of a protective group such as for example methyloxycarbonyl, benzyl or trialkylsilyl groups.

For those compounds of formula I wherein R¹ or R² represents thiazolyl or benzthiazolyl the secondary amines are prepared according to reaction scheme 5. In a first step the aminopiperidine of formula (vii) is converted into the intermediate of formula (ix) by reaction with an isothiocyanate of formula (viii) under art known reaction conditions (see scheme 2 above). For those intermediates where Rⁱⁱ represents hydrogen, the compounds of formula (I) are subsequently prepared by the cyclodesulfurization reaction of the thiourea derivative of formula (ix) by the reaction of (ix) with an appropriate alkyl halide (x) in an appropriate reaction-inert organic solvent, e.g., a lower alkanol such as methanol, ethanol, 2-propanol and the like. For those intermediates of formula (ix) where Rⁱⁱ does represent optionally substituted phenyl, the cyclodesulfurization reaction is carried out according to art-known procedures, such as for example using bromine in an aqueous hydrobromic acid solution.

Subsequently eliminating the protective group in the thus obtained intermediates of formula (xi) and (xi′) respectively, provides the appropriate secondary amines used as intermediates in the synthesis of the dimeric compounds of the present invention. The elimination of the protective group P in (xi, xi′) may generally be carried out following art-known procedures such as, for example, by hydrolysis in alkaline or acidic aqueous medium.

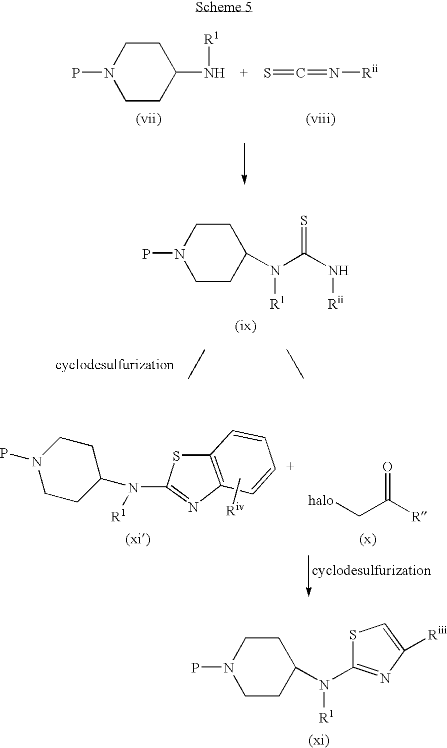

Wherein halo represents a halogen such as for example Cl, Br and I; R¹ is defined as for the compounds of formula (I); Rⁱⁱ represents hydrogen or an optionally substituted phenyl substituent; Rⁱⁱⁱ and R^{iv} each independently represent hydroxy, halo, Ar⁴, C₁₋₄alkyloxycarbonyl-, C₁₋₄alkyl-, C₁₋₄alkyloxy- or C₁₋₄alkyloxy- substituted with halo, wherein Ar⁴ is defined as for the compounds of formula (I)

Alternatively, the appropriate secondary amines are prepared by reductive amination from the piperidone (xii) with an amine of general formula (xiii) to yield the intermediate of formula (xiv). Further substitution of the secondary amine with an alkyl halide (xv) or acyl halide (xvi) under art known conditions (supra) provides the intermediates of formula (xvii) and (xvii′) respectively. Subsequently eliminating the protective group in the thus obtained intermediates, provides the appropriate secondary amines used as intermediates in the synthesis of the dimeric compounds of the present invention.

Alternatively the intermediate of formula (xiv) is converted into the thiourea derivative of formula (ix) by reaction with an isothiocyanate of formula (viii) under art known reaction conditions (see scheme 5 above). Subsequent cyclodesulfurization (supra) and deprotection (supra) provides the appropriate secondary amines.

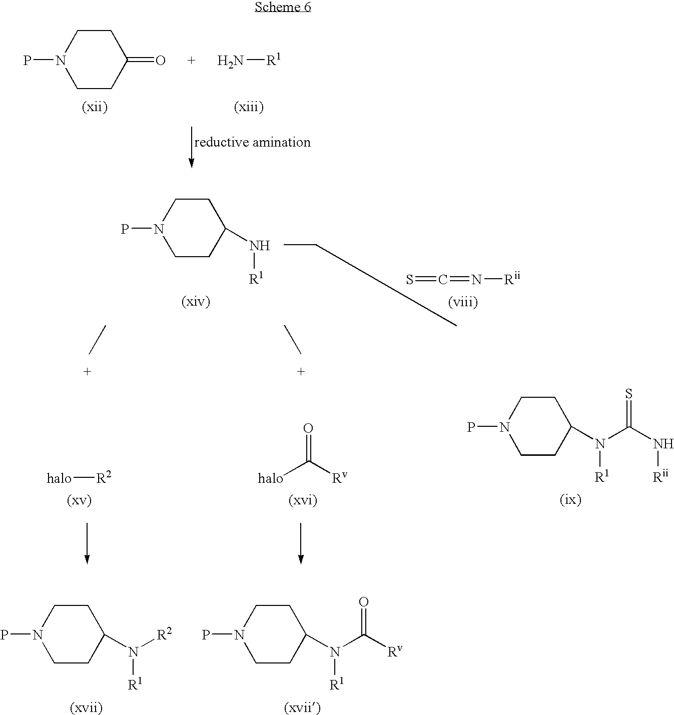

Wherein halo represents a halogen such as for example Cl, Br and I; R¹ and R² are defined as for the compounds of formula (I); Rⁱⁱ represents hydrogen or an optionally substituted phenyl substituent; R^{v} represent hydroxy, halo, Ar⁴, C₁₋₄alkyloxycarbonyl-, C₁₋₄alkyl-, C₁₋₄-alkyloxy- or C₁₋₄alkyloxy- substituted with halo, wherein Ar⁴ is defined as for the compounds of formula (I)

Further examples for the synthesis of compounds of formula (I) using anyone of the above mentioned synthesis methods, are provided in the experimental part hereinafter.

Where necessary or desired, any one or more of the following further steps in any order may be performed:
(i) removing any remaining protecting group(s);
(ii) converting a compound of formula (I) or a protected form thereof into a further compound of formula (I) or a protected form thereof;
(iii) converting a compound of formula (I) or a protected form thereof into a N-oxide, a salt, a quaternary amine or a solvate of a compound of formula (I) or a protected form thereof;
(iv) converting a N-oxide, a salt, a quaternary amine or a solvate of a compound of formula (I) or a protected form thereof into a compound of formula (I) or a protected form thereof;
(v) converting a N-oxide, a salt, a quaternary amine or a solvate of a compound of formula (I) or a protected form thereof into another N-oxide, a pharmaceutically acceptable addition salt a quaternary amine or a solvate of a compound of formula (I) or a protected form thereof;

It will be appreciated by those skilled in the art that in the processes described above the functional groups of intermediate compounds may need to be blocked by protecting groups.

Functional groups which it is desirable to protect include hydroxy, amino and carboxylic acid. Suitable protecting groups for hydroxy include trialkylsilyl groups (e.g. tert-butyldimethylsilyl, tert-butyldiphenylsilyl or trimethylsilyl), benzyl and tetrahydropyranyl. Suitable protecting groups for amino include tert-butyloxycarbonyl or benzyloxycarbonyl. Suitable protecting groups for carboxylic acid include C(₁₋₆)alkyl or benzyl esters.

The protection and deprotection of functional groups may take place before or after a reaction step.

The use of protecting groups is fully described in ‘Protective Groups in Organic Chemistry’, edited by J W F McOmie, Plenum Press (1973), and ‘Protective Groups in Organic Synthesis’ 2^{nd} edition, T W Greene & P G M Wutz, Wiley Interscience (1991).

Additionally, the N-atoms in compounds of formula (I) can be methylated by art-known methods using CH₃—I in a suitable solvent such as, for example 2-propanone, tetrahydrofuran or dimethylformamide.

The compounds of formula (I), can also be converted into each other following art-known procedures of functional group transformation of which some examples are mentioned hereinabove.

The compounds of formula (I), may also be converted to the corresponding N-oxide forms following art-known procedures for converting a trivalent nitrogen into its N-oxide form. Said N-oxidation reaction may generally be carried out by reacting the starting material of formula (I) with 3-phenyl-2-(phenylsulfonyl)oxaziridine or with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. t-butyl hydroperoxide. Suitable solvents are, for example, water, lower alkanols, e.g. ethanol and the like, hydro-carbons, e.g. toluene, ketones, e.g. 2-butanone, halogenated hydrocarbons, e.g. dichloromethane, and mixtures of such solvents.

Pure stereochemically isomeric forms of the compounds of formula (I), may be obtained by the application of art-known procedures. Diastereomers may be separated by physical methods such as selective crystallization and chromatographic techniques, e.g. counter-current distribution, liquid chromatography and the like.

Some of the compounds of formula (I), and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials as used in the reaction procedures mentioned hereinabove are known compounds and may be commercially available or may be prepared according to art-known procedures.

The compounds of the present invention are useful because they possess pharmacological properties. They can therefore be used as medicines, in particular to treat pain, in particular post-operative pain and pathologies associated with neuronal death, such as, stroke, Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia, and other tauopathies. Further conditions involving neurodegenerative processes are for instance, age-related macular degeneration, narcolepsy, motor neuron diseases, prion diseases, traumatic nerve injury and repair, and multiple sclerosis.

As described in the experimental part hereinafter, the neurotrophic activity of the present compounds on p75 mediated neuronal death has been demonstrated in vitro, in an assay that determines the survival effect of the compounds on chick DRG neurons using the neurotrophic factor NGF as internal reference. This assay is based on a fluorimetric Calcein-AM measurement and addresses the functional response of neurons as a quantitative measure of survival.

Accordingly, the present invention provides the compounds of formula (I) and their pharmaceutically acceptable N-oxides, addition salts, quaternary armines and stereochemically isomeric forms for use in therapy. More particular in the treatment or prevention of neurodegenerative mediated disorders. The compounds of formula (I), and their pharmaceutically acceptable N-oxides, addition salts, quaternary amines and the stereochemically isomeric forms may hereinafter be referred to as compounds according to the invention.

In view of the utility of the compounds according to the invention, there is provided a method for the treatment of an animal, for example, a mammal including humans, suffering from a neurodegenerative disorder such as stroke, Alzheimer's disease, ALS, epilepsy, SCI, MS, MND and other neurodegenerative diseases as mentioned hereinbefore, which comprises administering an effective amount of a compound according to the present invention. Said method comprising the systemic or topical administration of an effective amount of a compound according to the invention, to warm-blooded animals, including humans.

It is thus an object of the present invention to provide a compound according to the present invention for use as a medicine. In particular to use the compound according to the present invention in the manufacture of a medicament for treating pathologies associated with neuronal death such as for example, stroke, Alzheimer's disease, ALS, epilepsy, SCI, MS, MND and other neurodegenerative diseases as mentioned hereinbefore.

In yet a further aspect, the present invention provides the use of the compounds according to the invention in the manufacture of a medicament for treating any of the aforementioned neurodegenerative disorders or indications.

The amount of a compound according to the present invention, also referred to here as the active ingredient, which is required to achieve a therapeutical effect will be, of course, vary with the particular compound, the route of administration, the age and condition of the recipient, and the particular disorder or disease being treated. A suitable daily dose would be from 0.001 mg/kg to 500 mg/kg body weight, in particular from 0.005 mg/kg to 100 mg/kg body weight. A method of treatment may also include administering the active ingredient on a regimen of between one and four intakes per day.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy, for example, using methods such as those described in Gennaro et al. Remington's Pharmaceutical Sciences (18^{th} ed., Mack Publishing Company, 1990, see especially Part 8: Pharmaceutical preparations and their Manufacture). A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for examples may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, gellies, dressings, shampoos, tinctures, pastes, ointments, salves, powders and the like. Application of said compositions may be by aerosol, e.g. with a propellant such as nitrogen, carbon dioxide, a freon, or without a propellant such as a pump spray, drops, lotions, or a semisolid such as a thickened composition which can be applied by a swab. In particular, semisolid compositions such as salves, creams, gellies, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

In order to enhance the solubility and/or the stability of the compounds of formula (I) in pharmaceutical compositions, it can be advantageous to employ α-, β- or γ-cyclo-dextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Experimental Part

Hereinafter, the term ‘RT’ means room temperature, ‘MIK’ means 4-methyl-2-pentanone, ‘THF’ means tetrahydrofuran, ‘DIPE’ means diisopropyl ether, ‘DMSO’ means dimethylsulfoxide.

### A. PREPARATION OF THE INTERMEDIATES

### EXAMPLE A1

a) Preparation of

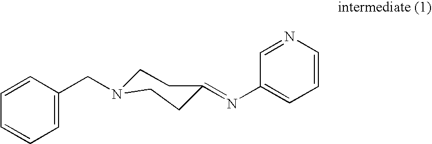

A mixture of 1-(phenylmethyl)-4-piperidinone (0.1 mol), 3-pyridinamine (0.125 mol) and 4-methylbenzenesulfonic acid (catalytic quantity) in toluene (150 ml) was stirred for 5 hours using a water separator. The solvent was evaporated. The residue (oil) was dissolved in DIPE, filtered and the filtrate's solvent was evaporated, yielding 27 g of intermediate (1).

b) Preparation of

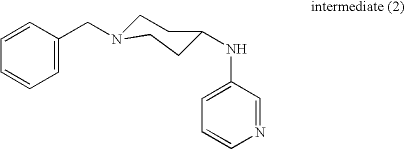

Intermediate (1) (0.1 mol) was stirred in ethanol (50 ml). Sodium tetrahydroborate (0.1 mol) was added and the reaction mixture was warmed to 50° C. Upon completion, the solvent was evaporated. The oily residue was stirred in 1 N HCl (150 ml), then filtered. The filtrate was alkalised with NH₄OH, then extracted with toluene. The separated organic layer was dried (MgSO₄), filtered and the solvent evaporated. The residue was washed with DIPE, then dried in vacuo, yielding 14 g of intermediate (2); m.p.±130° C.

c) Preparation of

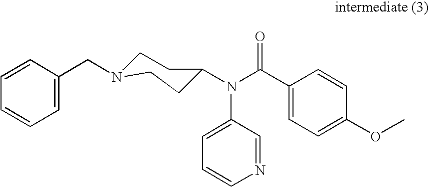

A mixture of intermediate (2) (0.4 mol) and N,N-diethylethanamine (1.6 mol) in benzene (2400 ml) was stirred in a 5-L reaction flask. A solution of 4-methoxybenzoyl chloride (0.8 mol) in benzene (1000 ml) was added dropwise (exothermic temperature rise). The reaction mixture was warmed gently to reflux temperature, then stirred and refluxed overnight. The mixture was cooled, filtered and the filtrate was evaporated. The residue was dissolved in MIK. This solution was washed with a diluted NaOH solution (2×), then with water (2×). The organic layer was separated, dried, filtered and the solvent was partially evaporated. The concentrate (±500 ml) was extracted three times with acidic water. The acidic water layer was extracted once with CHCl₃. The CHCl₃ layer was extracted three times with acidic water. All acidic water layers were combined, then washed 1× with DIPE. The water layer was alkalised with a dilute NaOH solution. The aqueous layers were extracted twice with CHCl₃. The separated organic layer was washed with water;, dried (MgSO₄), filtered and the solvent evaporated. The residue was crystallized from CH₃OH, filtered off and dried, yielding 22 g of intermediate (3).

d) Preparation of

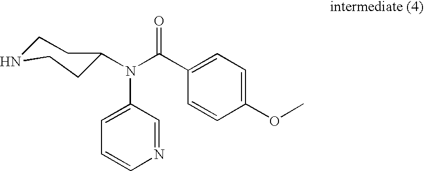

A mixture of intermediate (3) (0.18 mol) in methanol (500 ml) was hydrogenated with palladium on activated carbon (10%) (10 g) as a catalyst. After uptake of hydrogen (1 equiv.), the catalyst was filtered off and the filtrate was evaporated, yielding 62 g of intermediate (4).

### EXAMPLE A2

a) Preparation of

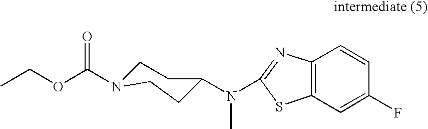

Bromine (0.3 mol) was added dropwise to a mixture of 4-[[[(4-fluorophenyl)amino]-thioxomethyl]methylamino]-1-piperidinecarboxylic acid, ethyl ester [104605-22-3] (0.3 mol) in tetrachloromethane (600 ml). The reaction mixture was stirred for one hour at room temperature, then it was heated to reflux temperature. The reaction mixture was stirred and refluxed for 3 hours (HBr gas evolution). The mixture was cooled. The solvent (CCl₄) was decanted off, yielding 101 g of intermediate (5) (quantitative yield; used in next reaction step, without further purification).

b) Preparation of

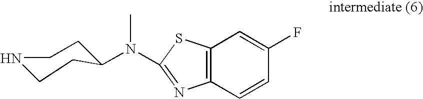

A mixture of intermediate (5) (0.3 mol) in a hydrobromic acid solution in water (48%) (800 ml) was stirred and refluxed for 6 hours, then stood over the weekend at room temperature. The solvent was evaporated. The residue was stirred in boiling 2-propanol, cooled and the resulting precipitate was filtered off and dried. The solid was dissolved in water (600 ml), alkalized with 50% NaOH, then extracted with dichloromethane. The separated organic layer was dried, filtered and the solvent evaporated. The residue was purified by column chromatography over silica gel (eluent 1: CH₂Cl₂/CH₃OH 98/2, then eluent 2: CHCl₃/CH₃OH/NH₄OH 85/10/5). The product fractions were collected and the solvent was evaporated, yielding 31 g (39%) of intermediate (6).

### EXAMPLE A3

a) Preparation of

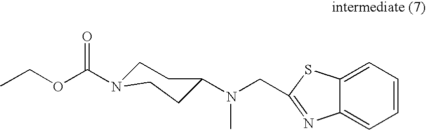

A mixture of 4-(methylamino)-1-piperidinecarboxylic acid, ethyl ester [73733-69-4] (0.2 mol), 2-(chloromethyl)benzothiazole [37859-43-1] (0.22 mol) and sodium carbonate (0.4 mol) in DMF (400 ml) was stirred overnight at 66° C., then the reaction mixture was poured out into ice water and extracted with dichloromethane. The organic layer was separated, dried, filtered off and the solvent was evaporated. The residue was purified by column chromatography (eluent: CH₂Cl₂/CH₃OH 99/1). The product fractions were collected and the solvent was evaporated. The obtained residue was crystallized from 2-propanol and the resulting precipitate was collected, yielding 32.5 g (48.7%) of intermediate (7); m.p. 101.9° C.

b) Preparation of

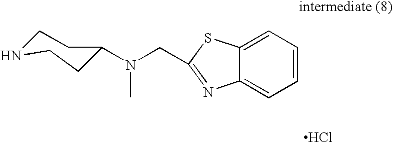

A mixture of intermediate (7) (0.05 mol) and potassium hydroxide (0.5 mol) in 2-propanol (350 ml) was stirred and refluxed for 5 hours and then the solvent was evaporated. Water was added to the residue and the resulting mixture was extracted with dichloromethane. The organic layer was separated, dried, filtered off and the solvent was evaporated. The obtained residue was dissolved in 2-propanol and acidified with HCl/2-propanol and then the resulting hydrochloric acid salt (1:2) was collected, yielding 6.6 g (38.4%) of intermediate (8); m.p. 205.0° C.

### EXAMPLE A4

a) Preparation of

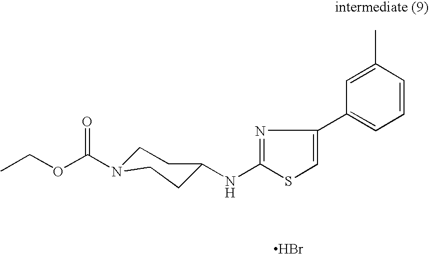

A mixture of 4-[(aminothioxomethyl)amino]-1-piperidinecarboxylic acid, ethyl ester [294622-57-4] (0.1 mol) and 2-bromo-1-(3-methylphenyl)ethanone [51012-64-7] (0.11 mol) in ethanol (300 ml) was stirred and refluxed overnight. The solvent was evaporated. The residue was washed with DIPE, yielding 42.6 g of intermediate (9) (quantitative yield; used in next reaction step, without further purification).

b) Preparation of

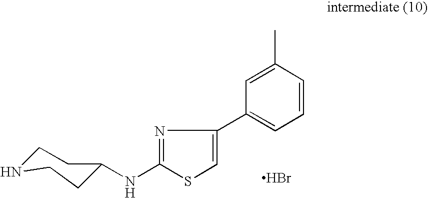

A mixture of intermediate (9) (0.1 mol) in hydrobromic acid (48%) (200 ml) was stirred and refluxed for 30 minutes, then allowed to cool and crystallize out while stirring. The precipitate was filtered off, washed with 2-propanone/DIPE, filtered off and dried, yielding 33 g of intermediate (10); m.p. 258° C.

### EXAMPLE A5

a) Preparation of

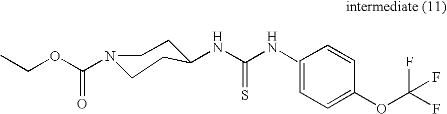

4-(trifluoromethoxy)benzenamine (0.141 mol) dissolved in THF (50 ml) was added dropwise to a solution of 4-isothiocyanato-1-piperidinecarboxylic acid, ethyl ester [73733-70-7] (0.15 mol) in THF (200 ml) and the mixture was stirred at room temperature overnight. The precipitate was filtered off and dried, yielding 51.6 g (93.5%) of intermediate (11); m.p. 133.2° C.

b) Preparation of

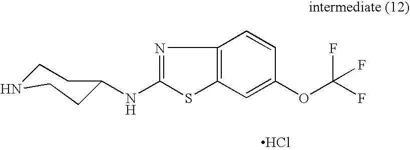

Bromine (0.05 mol) was added dropwise (slowly) at 50° C. to a mixture of intermediate (11) (0.05 mol) in a solution of hydrobromic acid in water (48%) (150 ml). The mixture was warmed up till reflux and stirred and refluxed for 6 hours. The mixture was cooled with stirring and crystallized. The precipitate was filtered off and dried. The filtrate was evaporated, taken up in water, alkalized with NH₄OH and extracted with dichloromethane. The organic layer was dried, filtered off and evaporated. The residue was dissolved in 2-propanone and converted into the hydrochloric acid salt (1:2) in 2-propanol, yielding 1.8 g (9.2%) of intermediate (12); m.p. 259° C.

### EXAMPLE A6

a) Preparation of

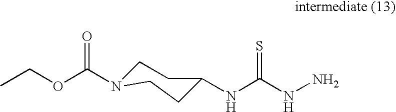

Hydrazine monohydrate(0.1 mol) was added dropwise to a mixture of 4-isothiocyanato-1-piperidinecarboxylic acid ethyl ester [73733-70-7] (0.05 mol) in THF (200 ml) and the reaction mixture was stirred overnight at room temperature, then the mixture was stirred and refluxed for 30 minutes. After cooling, the resulting precipitate was filtered off and dried, yielding 8.8 g (71.9%) of intermediate (13).

b) Preparation of

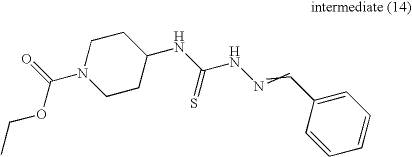

A mixture of intermediate (13) (0.1 mol) and benzaldehyde (0.1 mol) in ethanol (200 ml) was stirred and refluxed overnight and then the solvent was evaporated, yielding 33.5 g (100%) of intermediate (14).

c) Preparation of

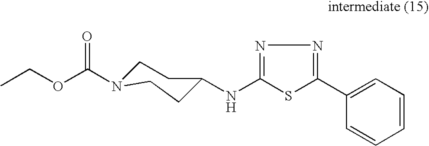

A mixture of intermediate (14) (0.1 mol) and Iron chloride. hydrate (1:6) (0.36 mol) in water (300 ml) was stirred and refluxed over the weekend and the solvent was evaporated. The residue was neutralized with a 10% K₂CO₃ solution and the resulting mixture was extracted with dichloromethane. The organic layer was separated, dried, filtered off and the solvent was evaporated, yielding 28.6 g (86%) of intermediate (15).

d) Preparation of

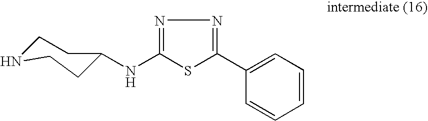

A mixture of intermediate (15) (0.0255 mol) in hydrobromic acid (48%) (100 ml) was stirred and refluxed for 30 minutes and the solvent was evaporated. The residue was converted into the free base with NH₄OH and was extracted with dichloromethane. The organic layer was separated, dried, filtered off and the solvent was evaporated, yielding 6 g (90.2%) of intermediate (16).

### EXAMPLE A7

a) Preparation of

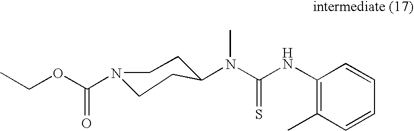

A solution of 1-isothiocyanato-2-methylbenzene (0.185 mol) in DIPE (100 ml) was added dropwise to a solution of 4-(methylamino)-1-piperidinecarboxylic acid, ethyl ester [73733-69-4] (0.185 mol) in DIPE (200 ml). The reaction mixture was stirred for 3 hours. The resulting precipitate was filtered off and dried, yielding 53.6 g (86.5%) of intermediate (17).

b) Preparation of

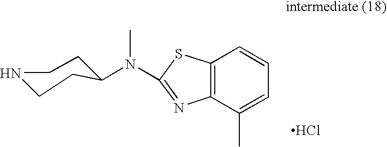

Bromine (0.165 mol) was added dropwise to intermediate (17) (0.16 mol) in hydrobromic acid (48%) (272 ml), stirred at 60° C. The reaction mixture was heated to reflux temperature, then stirred and refluxed overnight. The solvent was evaporated. The residue was treated with 50% NaOH and extracted with dichloromethane. The organic layer was separated, dried, filtered and the solvent evaporated. The residue was purified by column chromatography over silica gel (eluent: CHCl₃/CH₃OH 95/5). The product fractions were collected and the solvent was evaporated, yielding 30 g of product. Part (4.0 g) of the free base was dissolved in 2-propanone and converted into the hydrochloric acid salt (1:2) with HCl/2-propanol. The precipitate was filtered off and dried, yielding 2.5 g of intermediate (18); m.p. 295.5° C.

### EXAMPLE A8

a) Preparation of

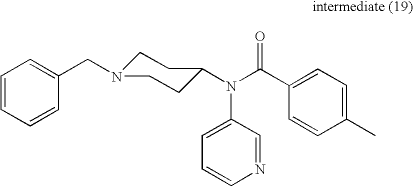

A mixture of N-[1-(phenylmethyl)-4-piperidinyl]-3-pyridinamine [63260-34-4] (0.2 mol) and N,N-diethylethanamine (0.8 mol) in benzene (1200 ml) was stirred at room temperature. A solution of 4-methyl benzoyl chloride (0.4 mol) in benzene (500 ml) was added dropwise (slightly exothermic reaction) and the resultant reaction mixture was heated slowly to reflux temperature. The mixture was stirred and refluxed for 12 hours, then cooled, filtered and the filtrate was evaporated. The residue was dissolved in CHCl₃. The organic solution was washed 3× with a 10% aqueous NaOH solution, twice with water, dried (MgSO₄), filtered and the solvent was evaporated. The residue was dissolved in an HCl solution 1/4, then stirred for a while. The acidic mixture was washed once with CHCl₃. The CHCl₃ layer was extracted three times with acidic water. The water layers were combined, washed 1× with DIPE, then alkalized with a 20% aqueous NaOH solution. This mixture was extracted three times with CHCl₃. The combined organic layers were washed with water, dried (MgSO₄), filtered and the solvent was evaporated, yielding 61 g of product. Part (4 g) of this product was recrystallized from 2-propanol, filtered off and dried, yielding 3 g of intermediate (19); m.p. 147.2° C.

b) Preparation of

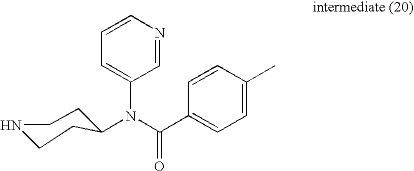

A mixture of intermediate (19) (0.16 mol) in methanol (500 ml) was hydrogenated with palladium on activated carbon (10%) (5 g) as a catalyst. After uptake of hydrogen (1 equiv.), the catalyst was filtered off and the filtrate was evaporated. Part (5 g) of the residue (47 g) was crystallized from 2-propanone/DIPE 1/10, filtered off and dried, yielding 4 g of intermediate (20); m.p. 137.2° C.

### EXAMPLE A9

a) Preparation of

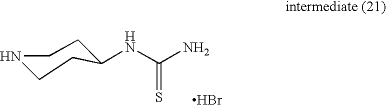

A mixture of 4-[(aminothioxomethyl)amino]-1-piperidinecarboxylic acid, ethyl ester [294622-57-4] (0.1 mol) in hydrobromic acid (48%) (200 ml) was stirred and refluxed for 2 hours. The mixture was allowed to cool to room temperature and crystallization resulted. The precipitate was filtered off, washed with DIPE and dried, yielding 15.1 g (47%/) of intemediate (21).

b) Preparation of

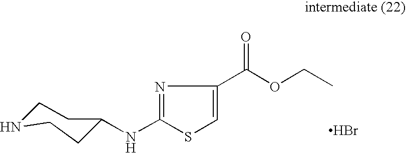

A suspension of intermediate (21) (0.05 mol)in ethanol (200 ml) was heated to reflux temperature. At reflux, 3-bromo-2-oxo-propanoic acid, ethyl ester (0.05 mol) was added dropwise (complete dissolution resulted). The reaction mixture was stirred and refluxed overnight. The mixture was allowed to cool to room temperature while stirring. Crystallization resulted and the precipitate was filtered off and dried, yielding 17.6 g (84.4%) of intermediate (22); m.p. 236.5° C.

### B. PREPARATION OF THE COMPOUNDS

### EXAMPLE B1

A mixture of intermediate (4) (0.0066 mol), 1,4-dichloro-2-butyne (0.0033 mol) and sodium carbonate (0.68 g) in MIK (20 ml) was stirred overnight at 100° C. The reaction mixture was washed with water (10 ml), and the organic solvent was evaporated. The residue was purified by HPLC over Kromasil silica gel (200 g, 100 Å, 5 μm) (eluent: CH₂Cl₂/(CH₂Cl₂/CH₃OH 90/10)/CH₃OH. The pure fractions were collected and the solvent was evaporated, yielding 0.94 g of product. This product was dried, yielding 0.492 g of compound 1.

### EXAMPLE B2

A mixture of N-methyl-N-4-piperidinyl-2-benzothiazolamine (0.0005 mol) and 1,4-diisocyanatobutane (0.5 equiv.) in dichloromethane (5 ml) was stirred overnight at room temperature. The desired compound was isolated and purified by column chromatography over silica gel (eluent: CH₂Cl₂/CH₃OH gradient from 100/0 to 90/10). The purest fractions were collected and the solvent was evaporated, yielding 0.062 g of compound 2.

### EXAMPLE B3

A mixture of 5-fluoro-N-methyl-N-4-piperidinyl-2-benzothiazolamine (0.01 mol) and N,N-diethylethanamine (0.012 mol) in dichloromethane(50 ml) was stirred at 0° C. Octanedioyl dichloride (0.005 mol) was added dropwise and the mixture was allowed to warm to room temperature. Stirring was continued overnight. Water was added and this mixture was extracted with dichloromethane. The separated organic layer was dried, filtered and the solvent evaporated. The residue was stirred in DIPE, filtered off and dried, yielding 1.67 g (50%) of compound 3.

### EXAMPLE B4

A solution of 1,3-dihydro-1-methyl-3-(4-piperidinyl)-2H-benzimidazol-2-one (0.0005 mol) in dichloromethane (2 ml) was mixed with a solution of N,N-diethylethanamine (0.0006 mol) in dichloromethane (1 ml). This mixture was treated dropwise with a solution of 4,4′-oxybisbenzenesulfonyl chloride (0.00025 mol) in THF (1 ml) and the resulting reaction mixture was stirred overnight under atmospheric conditions. The desired compound was isolated and purified by high-performance liquid chromatography over Kromasil Spherical underivated silica gel (55 g, 60 Å, 5 μm; eluent: CH₂Cl₂/(CH₂Cl₂/CH₃OH 9/1)/CH₃OH. The desired fractions were collected and the solvent was evaporated, yielding 0.140 g of compound 4.

Table F-1 lists the compounds that were prepared according to one of the above Examples.

TABLE F-1

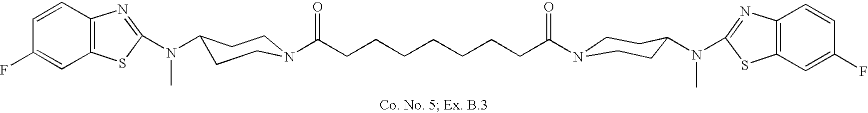

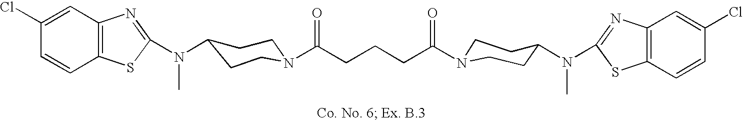

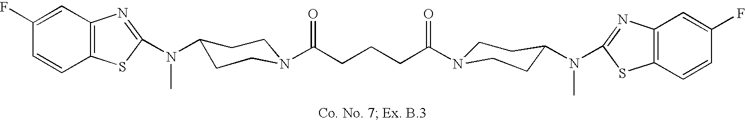

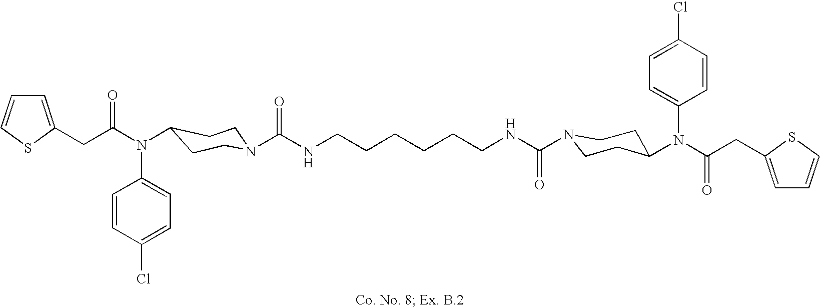

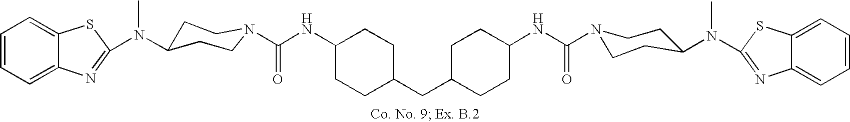

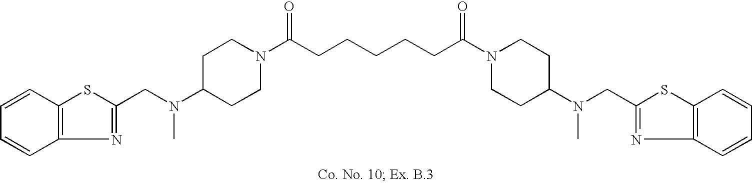

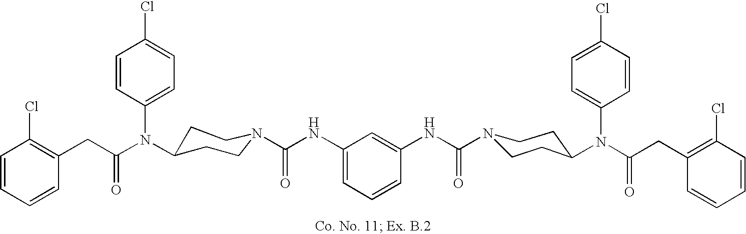

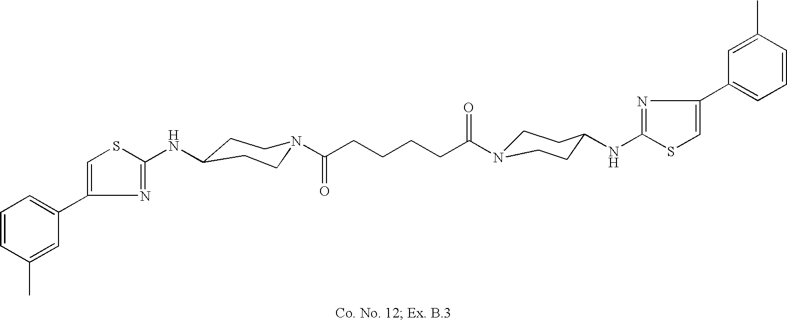

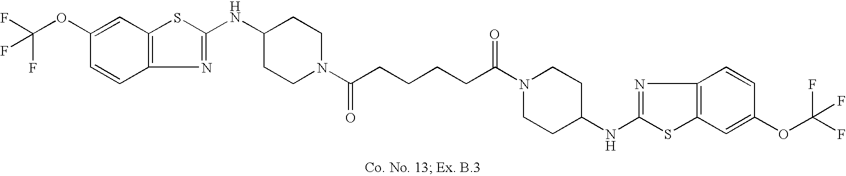

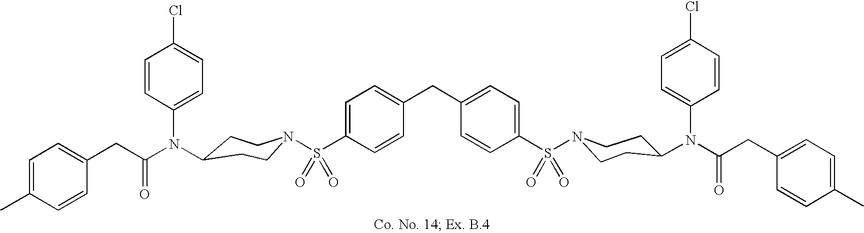

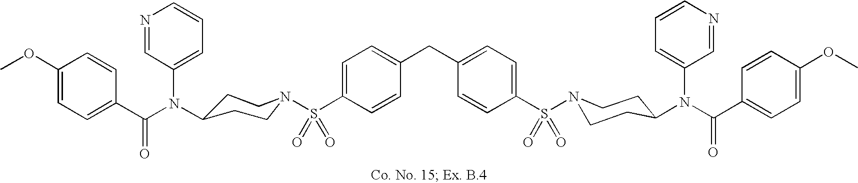

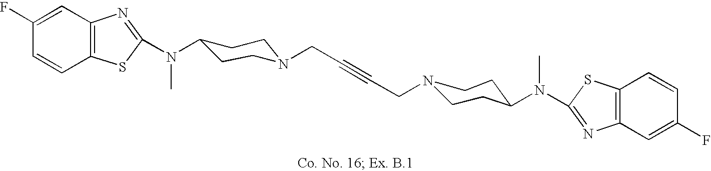

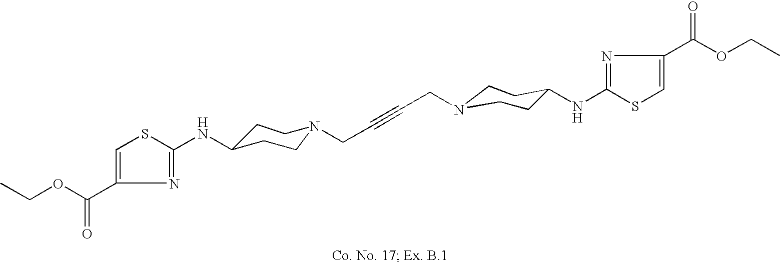

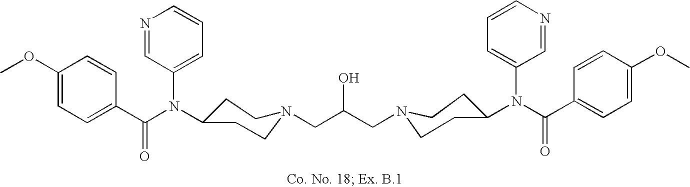

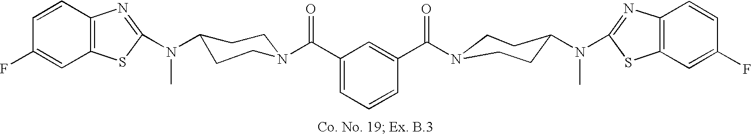

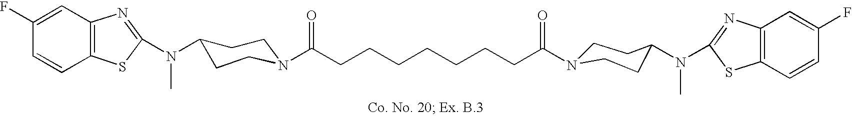

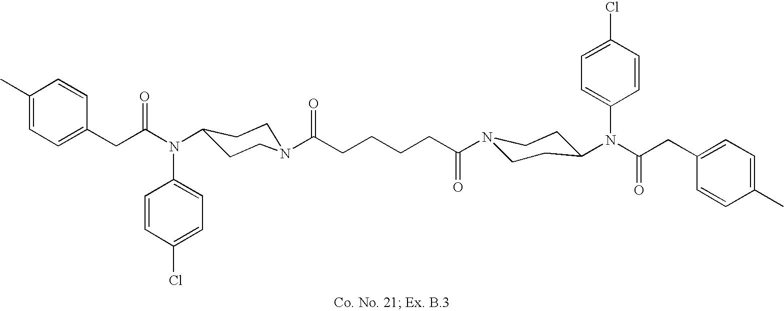

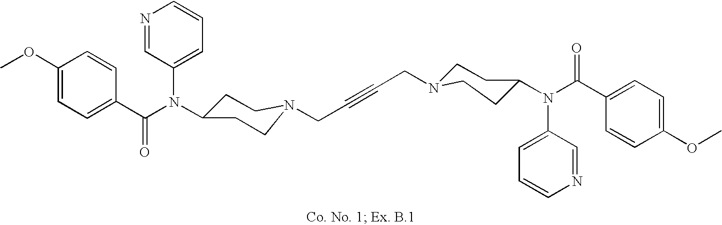

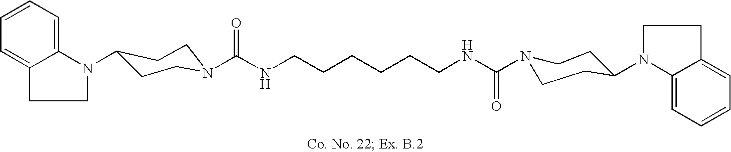

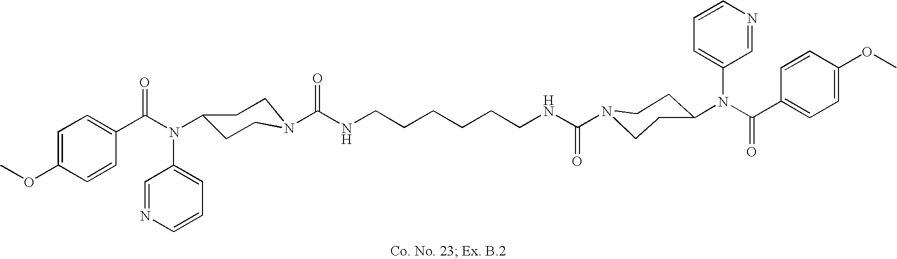

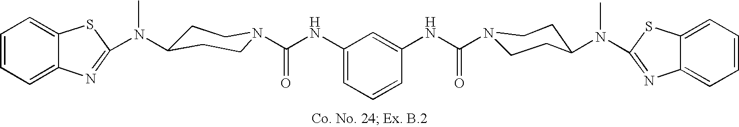

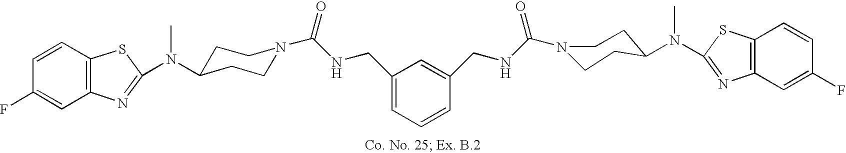

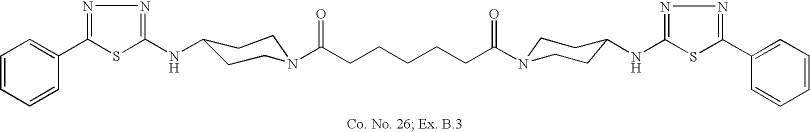

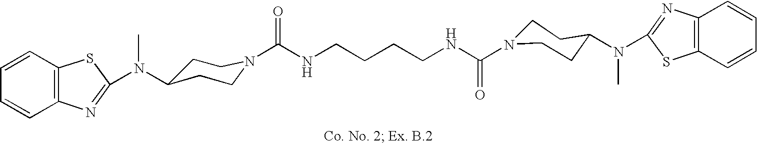

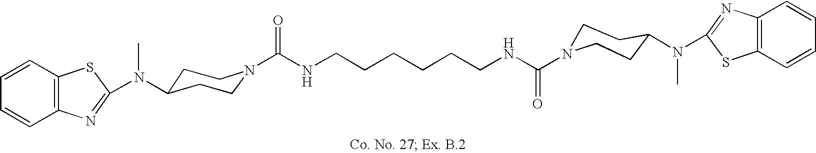

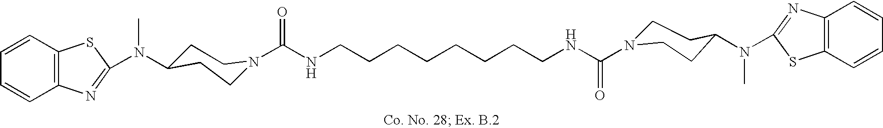

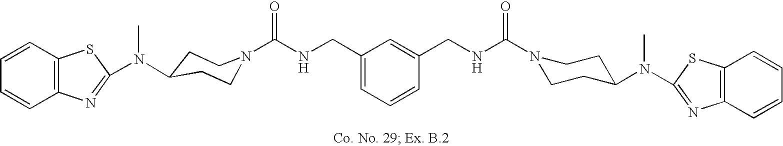

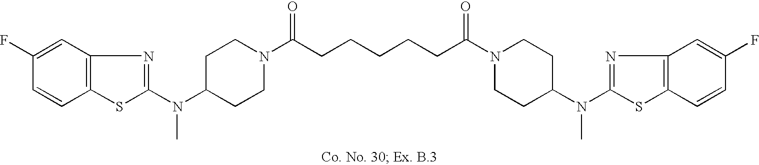

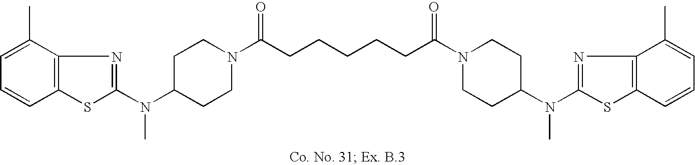

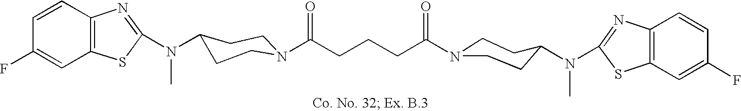

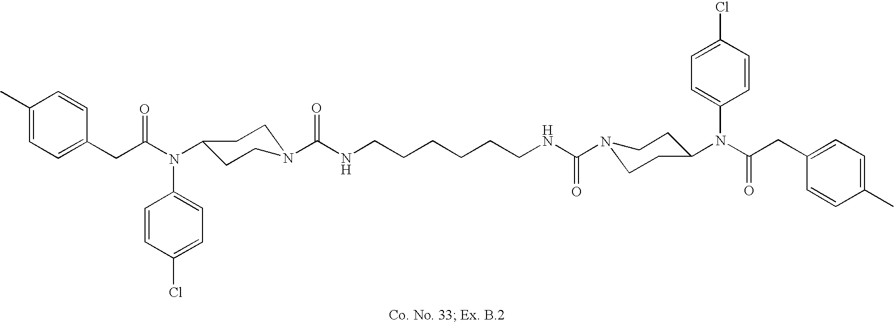

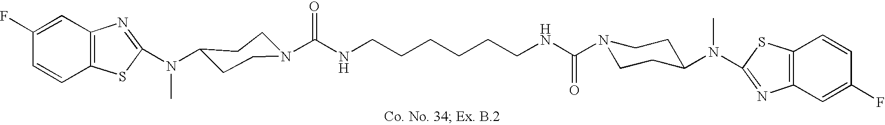

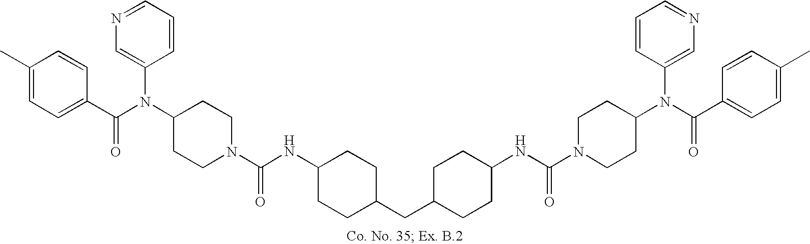

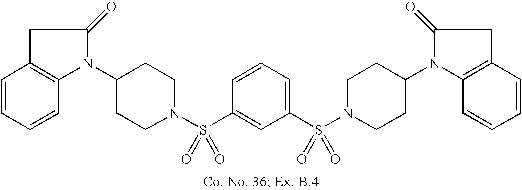

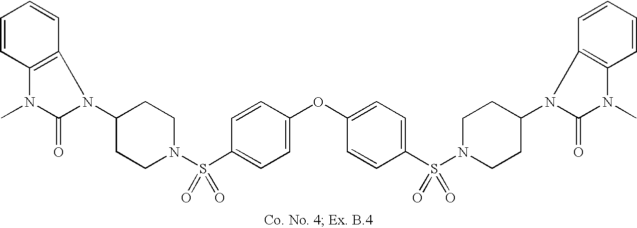

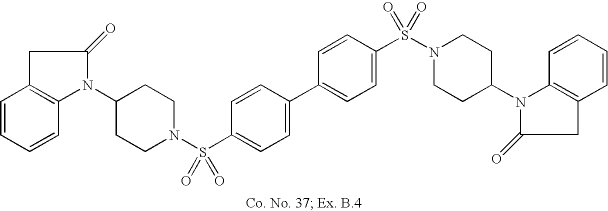

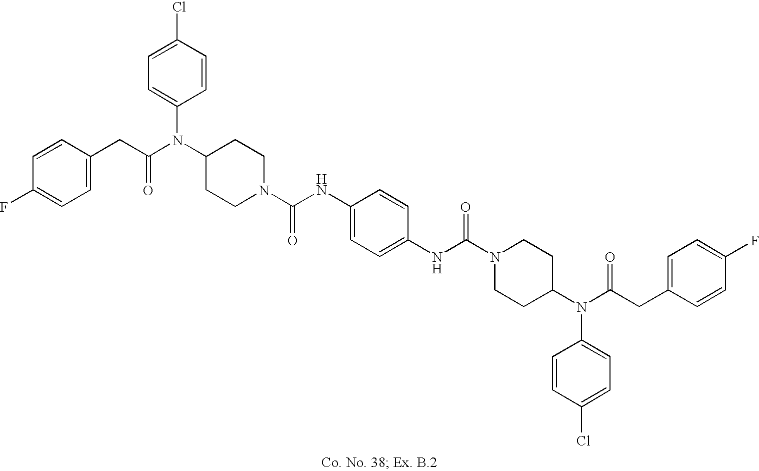

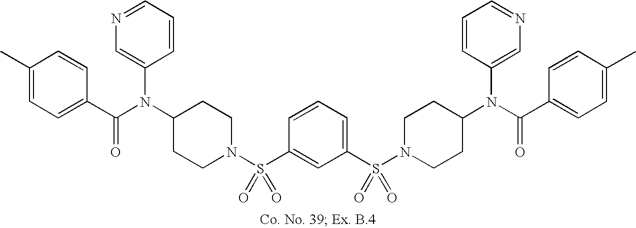

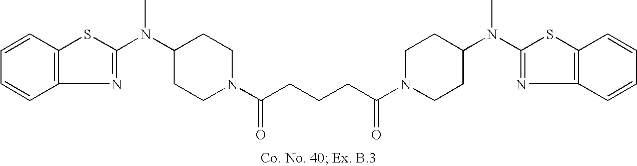

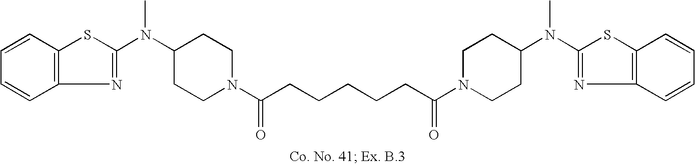

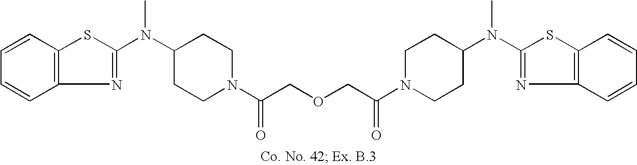

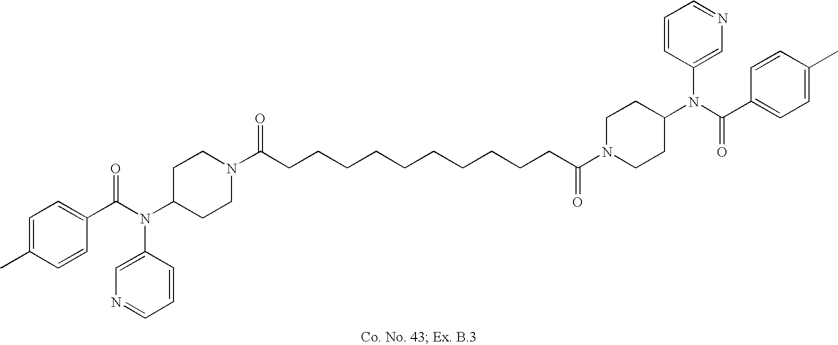

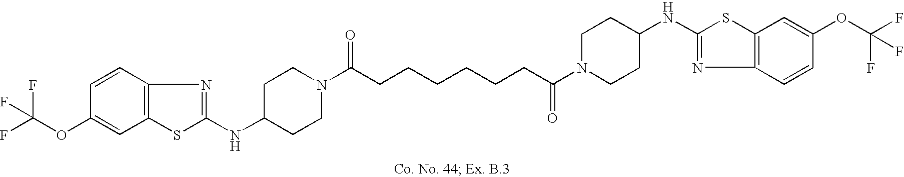

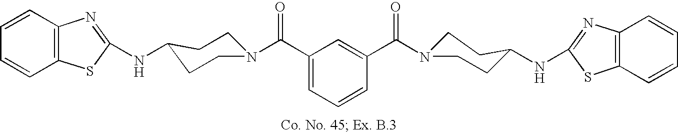

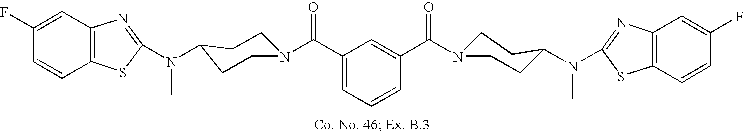

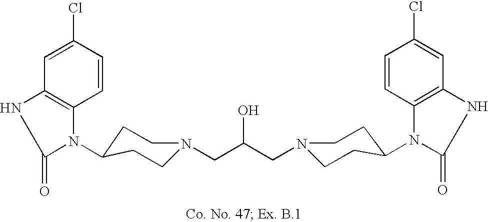

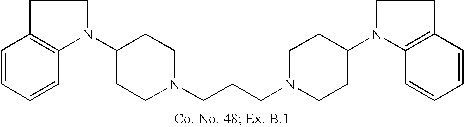

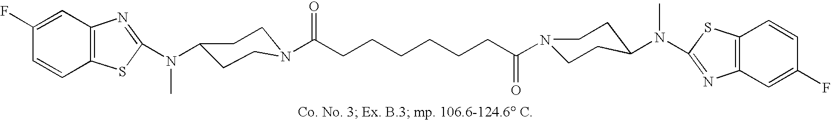

Compound Identification

The compounds were identified by LC/MS using a gradient elution system on a reversed phase HPLC. The compounds are identified by their specific retention time and their protonated molecular ion MH⁺ peak. The HPLC gradient was supplied by a Waters Alliance HT 2790 system with a columnheater set at 40° C. Flow from the column was split to a Waters 996 photodiode array (PDA) detector and a Waters-Micromass ZQ mass spectrometer with an electrospray ionization source operated in positive and negative ionization mode. Reversed phase HPLC was carried out on a Xterra MS C18 column (3.5 μm, 4.6×100 mm) with a flow rate of 1.6 ml/min. Three mobile phases (mobile phase A 95% 25 mM ammoniumacetate+5% acetonitrile; mobile phase B: acetonitrile; mobile phase C: methanol) were employed to run a gradient condition from 100% A to 50% B and 50% C in 6.5 minutes, to 100% B in 1 minute, 100% B for 1 minute and reequilibrate with 100% A for 1.5 minutes. An injection volume of 10 μL was used.

Mass spectra were acquired by scanning from 100 to 1000 in 1 s using a dwell time of 0.1 s. The capillary needle voltage was 3 kV and the source temperature was maintained at 140° C. Nitrogen was used a the nebulizer gas. Cone voltage was 10 V for positive ionization mode and 20 V for negative ionization mode. Data acquisition was performed with a Waters-Micromass MassLynx-Openlynx data system.

TABLE
retention time (RT in minutes) and molecular weight as the MH⁺

| Compound No. | Rt | MH+ |
|---|---|---|
| 5 | 7.9 | 683 |
| 8 | 5.91 | 837 |
| 9 | 6.54 | 757 |
| 18 | 5.44 | 679 |
| 20 | 7.86 | 683 |
| 22 | 6.02 | 573 |
| 23 | 5.82 | 791 |
| 25 | 6.06 | 719 |
| 27 | 5.98 | 663 |
| 28 | 6.21 | 691 |
| 29 | 5.96 | 683 |
| 31 | 6.83 | 647 |
| 33 | 6.65 | 853 |
| 34 | 6.1 | 699 |
| 45 | 6.73 | 597 |
| 3 | 6.32 | 669 |

### C. PHARMACOLOGICAL EXAMPLES

### EXAMPLE C.1

### Neuronal Viability Assay

Primary Culture of Chicken Dorsal Root Ganglion Neurons

Dorsal root ganglia were dissected from White Leghorn chick embryos at embryonic day 10 as described previously (Skaper S. D. and Varon S. (1986) Brain Research 389, 39-46). The ganglia were trypsinised and dissociated by mild trituration in a HBSS buffer supplemented with 0.6% glucose and 0.08% trypsin. To remove non-neuronal cells by differential attachment to culture plastic, the ganglionic cell suspension was lo diluted to 2.5×10⁵ cells/ml and seeded on tissue culture plastic dishes at 10 ml per 100 mm dish. After 2 h preplating, unattached neurons were collected and resuspended into Basal Eagle Medium containing 10% FCS. To remove cell aggregates, the cell suspension was passed through a nylon mesh (50 μM) pore diameter. Neuron-enriched cell suspension was plated at 5×10⁴ cells/ml into poly-L-ornithine (100 μg/ml) and laminine (1 μg/ml) coated multiwell 96 plates. Compounds were dissolved in dimethyl sulfoxide and kept as a stock at −20° C. NGF and compounds were diluted in the culture medium and added to the cells immediately after plating. The final concentration of dimethyl sulfoxide in the test medium was 0.1%. After two days of incubation, neuronal viability was assessed with calcein-AM.

Neuronal Viability Assay Using Calcein-AM

Neuronal viability assay using calcein AM was performed as previously described (Bozyczko-Coyne D., McKenna B. W., Connors T. J., and Neff N. T. (1993) Journal of Neuroscience Methods 50, 205-216). For the assay, calcein-AM was diluted in PBS to the final concentration (1 μM). For each experiment an aliquot of calcein-AM (1 mg/ml in DMSO stored at −20° C.) was thawed immediately before use. The medium was removed from the wells and replaced with the calcein-AM solution. Assay plates were incubated for 1 h at 37° C. in a humidified CO₂ incubator. Following the incubation, reading was done in a Cytofluor II at an excitation wavelength of 485 nm and an emission wavelength of 530 nm. Each plate had control wells with no neurotrophic factor added (0% survival) and wells with 10 ng/ml NGF (100% survival).

The drugs to be tested were taken from a stock solution and tested at a final concentration ranging from −10⁻⁵M to 3.10⁻⁹M. From the thus obtained dose response curves, the pIC50 value was calculated and scored as follows; Score 1=pIC50 value<6, Score 2=pIC50 value in the range of 6 to 8, Score 3=pIC50 value>8. Some of the thus obtained results are summarized in the table below.

| | [C1] DRG assay |
|---|---|
| Compound Number | SCORE |
| 1 | 2 |
| 2 | 2 |
| 3 | 2 |
| 4 | 2 |
| 5 | 2 |
| 6 | 2 |
| 7 | 2 |
| 8 | 2 |
| 9 | 2 |
| 10 | 1 |
| 11 | 2 |
| 12 | 2 |
| 13 | 2 |
| 14 | 2 |
| 15 | 1 |
| 16 | 2 |
| 17 | 2 |
| 18 | 2 |
| 19 | 2 |
| 20 | 2 |
| 21 | 2 |
| 22 | 2 |
| 23 | 2 |
| 24 | 2 |
| 25 | 2 |
| 26 | 1 |
| 27 | 2 |
| 28 | 1 |
| 29 | 2 |
| 30 | 2 |
| 31 | 1 |
| 32 | 2 |
| 33 | 2 |
| 34 | 2 |
| 35 | 2 |
| 36 | 1 |
| 37 | 2 |
| 38 | 2 |
| 39 | 2 |
| 40 | 1 |
| 41 | 2 |
| 42 | 1 |
| 43 | 2 |
| 44 | 2 |
| 45 | 2 |
| 46 | 1 |
| 47 | 1 |
| 48 | 2 |

### D. COMPOSITION EXAMPLES

The following formulations exemplify typical pharmaceutical compositions suitable for systemic or topical administration to animal and human subjects in accordance with the present invention.

“Active ingredient” (A.I.) as used throughout these examples relates to a compound of formula (D or a pharmaceutically acceptable addition salt thereof.

### EXAMPLE D.1

### Film-Coated Tablets

Preparation of Tablet Core

A mixture of A.I. (100 g), lactose (570 g) and starch (200 g) was mixed well and thereafter humidified with a solution of sodium dodecyl sulfate (5 g) and polyvinyl-pyrrolidone (10 g) in about 200 ml of water. The wet powder mixture was sieved, dried and sieved again. Then there was added microcrystalline cellulose (100 g) and hydrogenated vegetable oil (15 g). The whole was mixed well and compressed into tablets, giving 10,000 tablets, each comprising 10 mg of the active ingredient.

Coating

To a solution of methyl cellulose (10 g) in denaturated ethanol (75 ml) there was added a solution of ethyl cellulose (5 g) in CH₂Cl₂ (150 ml). Then there were added CH₂Cl₂ (75 ml) and 1,2,3-propanetriol (2.5 ml). Polyethylene glycol (10 g) was molten and dissolved in dichloromethane (75 ml). The latter solution was added to the former and then there were added magnesium octadecanoate (2.5 g), polyvinyl-pyrrolidone (5 g) and concentrated color suspension (30 ml) and the whole was homogenated. The tablet cores were coated with the thus obtained mixture in a coating apparatus.

1. A compound having the formula 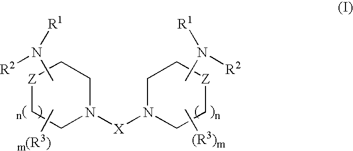
the N-oxide forms, the pharmaceutically acceptable addition salts and the stereochemically isomeric forms thereof, wherein
n represents 1 or 2;
m represents 0, 1, 2 or 3;
Z represent C, N or O;
—X— represents C₂₋₄alkynyl, C₁₋₁₂alkyl optionally substituted with hydroxy or X represents a divalent radical of the formula 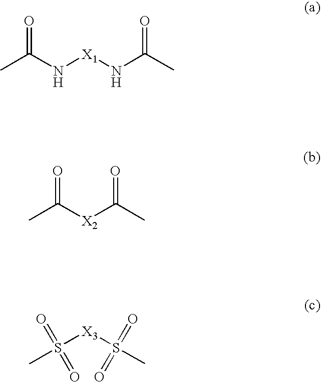
wherein; —X₁— represents C₁₋₁₂alkyl, phenyl or a divalent radical selected from the group consisting of 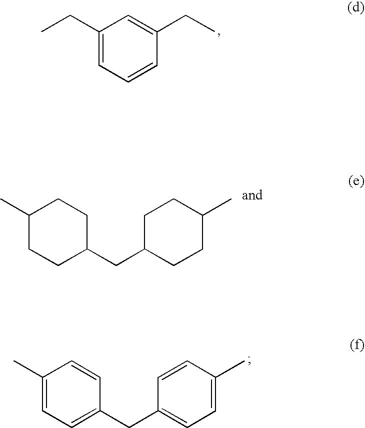
—X₂— represents C₁₋₁₂alkyl, C₁₋₄alkyloxyC₁₋₄alkyl, phenyl or a divalent radical of formula 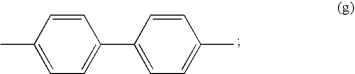
—X₃— represents phenyl or a divalent radical selected from the group consisting of 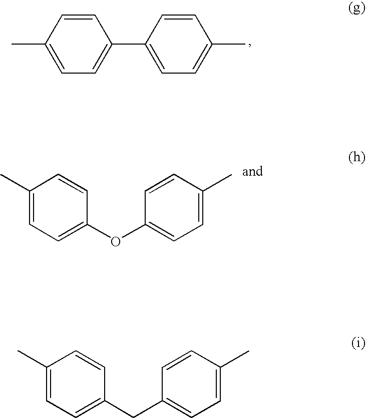
R¹ and R² each independently represents hydrogen, C₁₋₄alkyl, C₁₋₄alkyl-carbonyl-, Ar¹-carbonyl-, Het¹, Ar² or C₁₋₄alkyl-carbonyl- substituted with Het² or Ar³; or
R¹ and R² taken together with the nitrogen atom with which they are attached form a heterocycle selected from pyrimidinyl, indolyl, indolinyl, indazolyl, imidazolinyl, imidazolidinyl, benzoxazolyl, benzimidazolyl, quinazolinyl, quinolinyl or benzthiazolyl wherein said heterocycle is optionally substituted with one or where possible two or more substituents selected from the group consisting of carbonyl, Ar⁵, amino, mono- or di-substituted (C₁₋₄alkyl)-amino-, hydroxy, halo, polyhaloC₁₋₄alkyloxy-, C₁₋₄alkyl, C₁₋₄alkyloxycarbonyl- and phenyl;
R³ independently represents hydroxy or C₁₋₄alkyloxy-;
Het¹ represents a heterocycle selected from pyridinyl, indolinyl, benzimidazolyl, benzthiazolyl, thiazolyl, pyridinyl, benzisoxazolyl, benzoxazolyl, oxadiazolyl or thiadiazolyl wherein said Het¹ is optionally substituted with one or where possible two or more substituents selected from the group consisting of hydroxy, halo, Ar⁴, C₁₋₄alkyloxycarbonyl-, C₁₋₄alkyl-, C₁₋₄alkyloxy- and C₁₋₄alkyloxy- substituted with halo;
Het² represents a heterocycle selected from thiophenyl, furanyl, pyrrolyl, pyridinyl, thiazolyl, oxazolyl, pyridinyl, benzisoxazolyl, benzoxazolyl or thiadiazolyl wherein said Het² is optionally substituted with one or where possible two or more substituents selected from the group consisting of hydroxy, halo, Het⁴, C₁₋₄alkyloxycarbonyl-, C₁₋₄alkyl-, C₁₋₄alkyloxy- and C₁₋₄alkyloxy-substituted with halo;
Het³ represents a heterocycle selected from thiophenyl, furanyl, pyrrolyl, pyridinyl, thiazolyl, oxazolyl, pyridinyl, or thiadiazolyl;
Het⁴ represents a heterocycle selected from thiophenyl, furanyl, pyrrolyl, pyridinyl, thiazolyl, oxazolyl, pyridinyl, or thiadiazolyl wherein said Het⁴ is optionally substituted with one or where possible two or more substituents selected from the group consisting of hydroxy, halo, C₁₋₄alkyl- and C₁₋₄alkyloxy-;
Ar¹, Ar² and Ar³ each independently represent phenyl optionally substituted with halo, amino, Het³, C₁₋₄alkylcarbonyl-, C₁₋₄alkyl, C₁₋₄alkyloxy- or C₁₋₄alkyl substituted with one, two or three halo substituents; in particular Ar¹, Ar² and Ar³ each independently represent phenyl optionally substituted with halo, C₁₋₄alkyl or C₁₋₄alkyloxy-;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

2. A compound according to claim 1 wherein;
n represents 1;
m represents 0, 1 or 2; in particular m represents 0;
R¹ and R² each independently represent hydrogen, C₁₋₄alkyl, Ar¹-carbonyl, Het¹, Ar² or C₁₋₄alkylcarbonyl optionally substituted with Het² or Ar³; or
R¹ and R² taken together with the nitrogen atom to which they are attached form a heterocycle selected from indolyl, indolinyl, benzimidazolyl, benzthiazolyl, benzisoxazolyl or oxodiazolyl wherein said heterocycle is optionally substituted with one or where possible two or more substituents selected from the group consisting of hydroxy, C₁₋₄alkyl, carbonyl, C₁₋₄alkyloxycarbonyl-, Ar⁵ and halo;
Het¹ represents a heterocycle selected from pyridinyl, indolinyl, indolyl, benzthiazolyl, benzimidazolyl, thiazolyl, thiadiazolyl or benzisoxazolyl wherein said Het¹ is optionally substituted with one or where possible two or more substituents selected from the group consisting of halo, Ar⁴, C₁₋₄alkyloxycarbonyl-, C₁₋₄alkyl and C₁₋₄alkyloxy-, said C₁₋₄alkyloxy- being optionally substituted with halo;
Het² represents a heterocycle selected from thiophenyl, furanyl, pyrrolyl, pyridinyl, thiazolyl, oxazolyl, pyridinyl, or thiadiazolyl;
Ar¹, Ar² and Ar³ each independently represent phenyl optionally substituted with halo, C₁₋₄alkyl, C₁₋₄alkyloxy- or C₁₋₄alkyl substituted with one, two or three halo substituents;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

3. A compound according to claim 1 wherein;
n represents 1 or 2;
m represents 0, 1, 2 or 3;
Z represent CH₂;
—X— represents C₂₋₄alkynyl, C₁₋₁₂alkyl optionally substituted with hydroxy or X represents a divalent radical of the formula 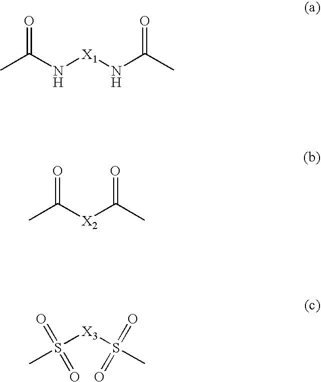
wherein; —X₁— represents C₁₋₁₂alkyl, phenyl or a divalent radical selected from the group consisting of 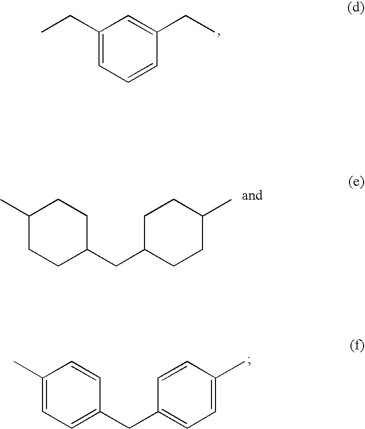
—X₂— represents C₁₋₁₂alkyl, C₁₋₄alkyloxyC₁₋₄alkyl, phenyl or a divalent radical of formula 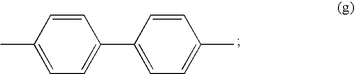
—X₃— represents phenyl or a divalent radical selected from the group consisting of 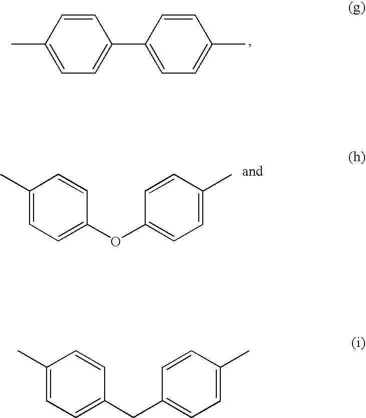
R¹ and R² each independently represents hydrogen, C₁₋₄alkyl, C₁₋₄alkyl-carbonyl-, Ar¹-carbonyl-, Het¹, Ar² or C₁₋₄alkyl-carbonyl-substituted with Het² or Ar³; or
R¹ and R² taken together with the nitrogen atom with which they are attached form a heterocycle selected from pyrimidinyl, indolyl, indolinyl, indazolyl, imidazolinyl, imidazolidinyl, benzoxazolyl, benzimidazolyl, quinazolinyl, quinolinyl or benzthiazolyl wherein said heterocycle is optionally substituted with one or where possible two or more substituents selected from the group consisting of carbonyl, Ar⁵, amino, mono- or di-substituted (C₁₋₄alkyl)-amino-, hydroxy, halo, polyhaloC₁₋₄alkyloxy-, C₁₋₄alkyl, C₁₋₄alkyloxycarbonyl- and phenyl;
R³ independently represents hydroxy or C₁₋₄alkyloxy-;
Het¹ represents a heterocycle selected from pyridinyl, indolinyl, benzimidazolyl, benzthiazolyl, thiazolyl, pyridinyl, benzisoxazolyl, benzoxazolyl, oxadiazolyl or thiadiazolyl wherein said Het¹ is optionally substituted with one or where possible two or more substituents selected from the group consisting of hydroxy, halo, Ar⁴, C₁₋₄alkyloxycarbonyl-, C₁₋₄alkyl-, C₁₋₄alkyloxy- and C₁₋₄alkyloxy-substituted with halo;
Het² represents a heterocycle selected from thiophenyl, furanyl, pyrrolyl, pyridinyl, thiazolyl, oxazolyl, pyridinyl, benzisoxazolyl, benzoxazolyl or thiadiazolyl wherein said Het² is optionally substituted with one or where possible two or more substituents selected from the group consisting of hydroxy, halo, Het⁴, C₁₋₄alkyloxycarbonyl-, C₁₋₄alkyl-, C₁₋₄alkyloxy- and C₁₋₄alkyloxy-substituted with halo;
Het³ represents a heterocycle selected from thiophenyl, furanyl, pyrrolyl, pyridinyl, thiazolyl, oxazolyl, pyridinyl, or thiadiazolyl;
Het⁴ represents a heterocycle selected from thiophenyl, furanyl, pyrrolyl, pyridinyl, thiazolyl, oxazolyl, pyridinyl, or thiadiazolyl wherein said Het⁴ is optionally substituted with one or where possible two or more substituents selected from the group consisting of hydroxy, halo, C₁₋₄alkyl- and C₁₋₄alkyloxy-;
Ar¹, Ar² and Ar³ each independently represent phenyl optionally substituted with halo, amino, Het³, C₁₋₄alkylcarbonyl-, C₁₋₄alkyl, C₁₋₄alkyloxy- or C₁₋₄alkyl substituted with one, two or three halo substituents; in particular Ar¹, Ar² and Ar³ each independently represent phenyl optionally substituted with halo, C₁₋₄alkyl or C₁₋₄alkyloxy-;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

4. A compound according to claim 1 wherein;
n represents 1;
m represents 0;
Z represents CH₂;
R¹ and R² each independently represent hydrogen, C₁₋₄alkyl, Ar¹-carbonyl, Het¹, Ar² or C₁₋₄alkylcarbonyl optionally substituted with Het² or Ar³; or
R¹ and R²taken together with the nitrogen atom to which they are attached form a heterocycle selected from indolyl, indolinyl, benzimidazolyl, benzthiazolyl, benzisoxazolyl or oxodiazolyl wherein said heterocycle is optionally substituted with one or where possible two or more substituents selected from the group consisting of hydroxy, C₁₋₄alkyl, carbonyl, C₁₋₄alkyloxycarbonyl-, Ar⁵ and halo;
Het¹ represents a heterocycle selected from pyridinyl, indolinyl, indolyl, benzthiazolyl, benzimidazolyl, thiazolyl, thiadiazolyl or benzisoxazolyl wherein said Het, is optionally substituted with one or where possible two or more substituents selected from the group consisting of halo, Ar⁴,
C₁₋₄alkyloxycarbonyl-, C₁₋₄alkyl and C₁₋₄alkyloxy-, said C₁₋₄alkyloxy- being optionally substituted with halo;
Het² represents a heterocycle selected from thiophenyl, furanyl, pyrrolyl, pyridinyl, thiazolyl, oxazolyl, pyridinyl, or thiadiazolyl;
Ar¹, Ar² and Ar³ each independently represent phenyl optionally substituted with halo, C₁₋₄alkyl, C₁₋₄alkyloxy- or C₁₋₄alkyl substituted with one, two or three halo substituents;
Ar⁴ represents phenyl optionally substituted with halo, C₁₋₄alkyl, C₁₋₄alkyloxy- or C₁₋₄alkyl substituted with one, two or three halo substituents;
Ar⁵ represents phenyl optionally substituted with C₁₋₄alkyloxy- or C₃₋₆cycloalkyloxy-.

5. A compound according to claim 1 wherein;
n represents 1;
m represents 0;
Z represents CH₂;
R¹ and R² each independently represents hydrogen, C₁₋₄alkyl, Ar¹-carbonyl-, Het¹, Ar² or C₁₋₄alkylcarbonyl-substituted with Het² or Ar³; or
R¹ and R² taken together with the nitrogen atom to which they are attached form a heterocycle selected from indolyl, indolinyl, or benzimidazolyl wherein said heterocycle is optionally substituted with one or where possible two or more substituents selected from the group consisting of carbonyl, hydroxy or halo;
Het¹ represents a heterocycle selected from pyridinyl, indolinyl, benzthiazolyl, thiazolyl, or thiadiazolyl, wherein said Het¹ is optionally substituted with one or where possible two or more substituents selected from the group consisting of halo, Ar⁴, C₁₋₄alkyloxycarbonyl- and C₁₋₄alkyloxy-substituted with halo;
Het² represents thiophenyl;
Ar¹ represents phenyl optionally substituted with halo or C₁₋₄alkyloxy-;
Ar² represents phenyl optionally substituted with halo or C₁₋₄alkyloxy;
Ar³ represents phenyl optionally substituted with halo or C₁₋₄alkyl; or
Ar⁴ represents phenyl optionally substituted with C₁₋₄alkyl-.

6. A compound according to claim 1 wherein;
m represents 0;
Z represents CH₂;
n represents 1;
—X— represents C₂₋₄alkynyl, C₁₋₁₂alkyl optionally substituted with hydroxy or —X— represents a divalent radical of the formula (a), (b) or (c) as defined hereinbefore
wherein; —X₁— represents C₁₋₁₂alkyl or a divalent radical selected from (d) or (e) as defined for the compounds of formula (I) hereinbefore;
—X₂— represents C₁₋₁₂alkyl, C₁₋₄alkyloxyC₁₋₄alkyl, phenyl or a divalent radical of formula (g) as defined for the compounds of formula (I) hereinbefore;
—X₃— represents phenyl or a divalent radical selected from the (g), (h) and (i) as defined for the compounds of formula (I) hereinbefore;
R¹ and R² each independently represent hydrogen, C₁₋₄alkyl or R¹ and R² taken together with the nitrogen atom to which they are attached form a heterocycle selected from indolyl, indolinyl or benzimidazolyl wherein said heterocycle is optionally substituted with one or where possible two or more substituents selected from the group consisting of carbonyl, hydroxy or halo;
Het¹ represents a heterocycle selected from pyridinyl, indolinyl or benzthiazolyl wherein said Het¹ is optionally substituted with halo, Ar⁴ or polyhaloC₁₋₄alkyloxy-;
Het² represents thiophenyl;
Ar¹ represents phenyl optionally substituted with halo or C₁₋₄alkyloxy-;
Ar² represents phenyl optionally substituted with halo or C₁₋₄alkyloxy;
Ar³ represents phenyl optionally substituted with halo or C₁₋₄alkyl; or
Ar⁴ represents phenyl optionally substituted with C₁₋₄alkyl-.

7. A compound according to claim 1 wherein;
Ar² represents phenyl substituted with halo

8. A compound as claimed in claim 1 wherein the compound is selected from the compounds with formulae (A)-(O) below:

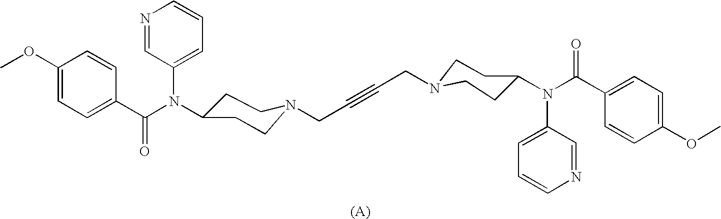

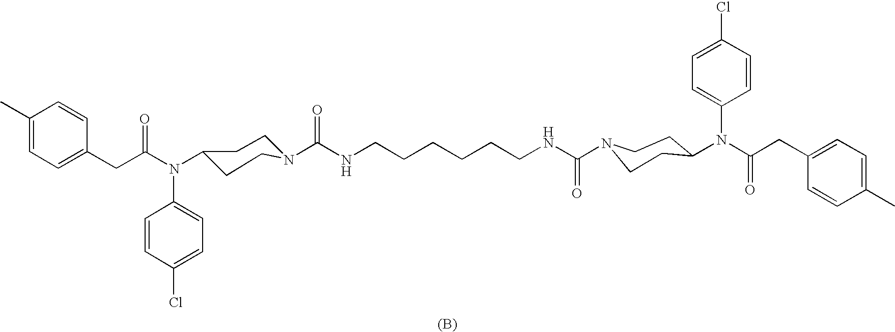

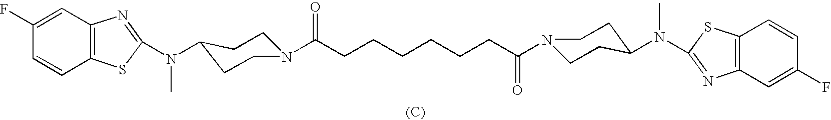

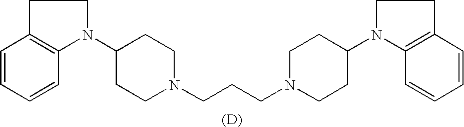

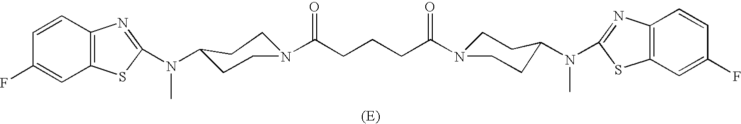

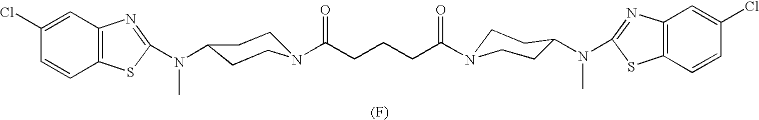

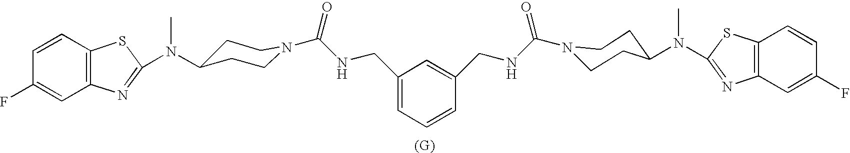

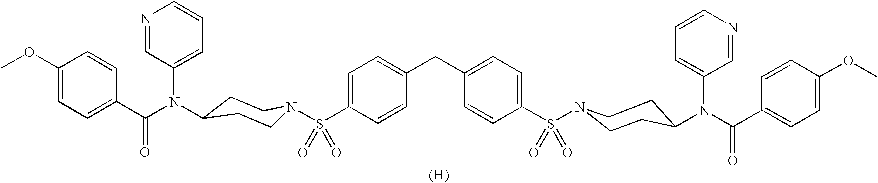

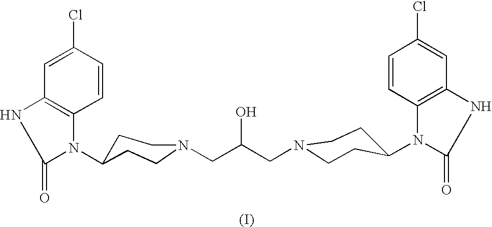

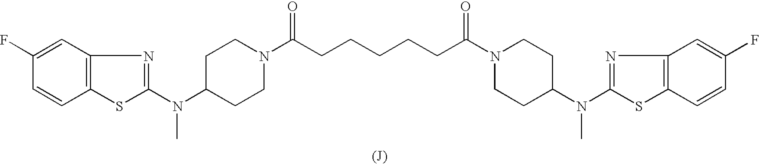

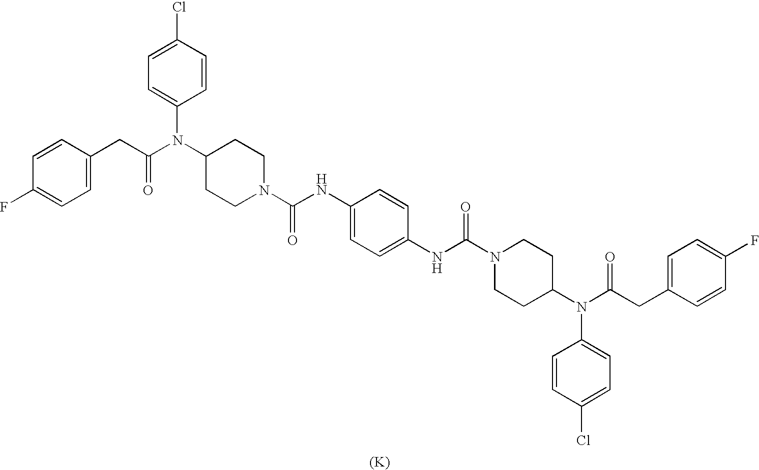

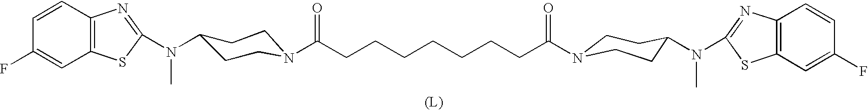

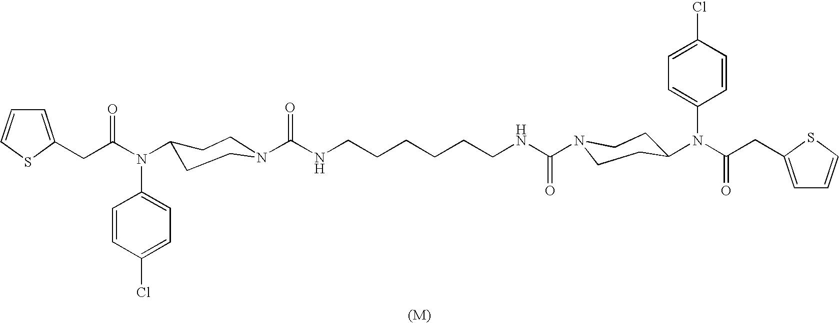

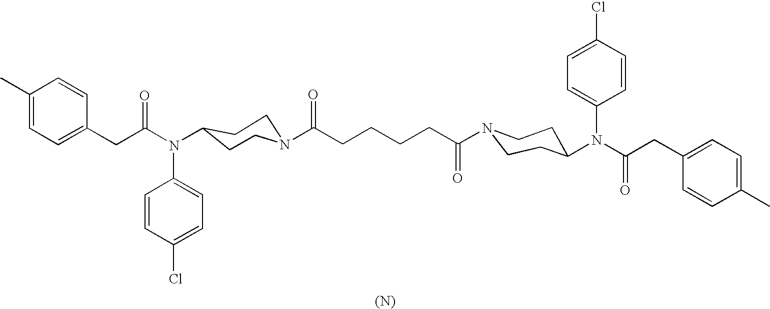

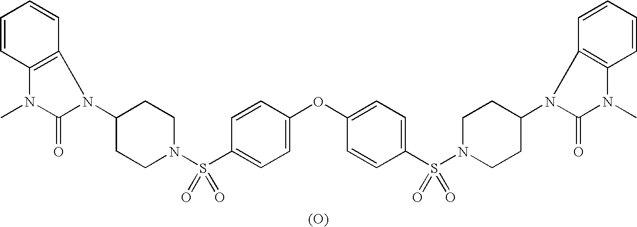

9. A pharmaceutical composition comprising a pharmaceutically acceptable carrier and, as active ingredient, a therapeutic effective amount of a compound as described in of claim 1.

10. (canceled)

11. (canceled)

12. The method of claim 13, wherein the pain is post-operative pain.

13. A method for treating pain comprising administering to a host in need thereof an effective amount of a compound as claimed in claim 1.

14. A method of treating pathologies associated with neuronal death, stroke, Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia, other tauopathies, and further conditions involving neurodegenerative processes are for instance, age-related macular degeneration, narcolepsy, motor neuron diseases, prion diseases, traumatic nerve injury and repair, and multiple sclerosis comprising administering to a host in need thereof an effective amount of a compound of claim 1.
